(19) United States
(12) Patent Application Publication
(10) Pub. No.: US 2024/0374786 A1
(43) Pub. Date: Nov. 14, 2024

(54) NANOFIBER AND MICROFIBER HYBRID AEROGELS

(71) Applicant: Board of Regents of the University of Nebraska, Lincoln, NE (US)

(72) Inventors: Jingwei Xie, Omaha, NE (US); Mark Carlson, Omaha, NE (US); S.M. SHATIL SHAHRIAR, Omaha, NE (US)

(21) Appl. No.: 18/600,263

(22) Filed: Mar. 8, 2024

Related U.S. Application Data
(60) Provisional application No. 63/464,732, filed on May 8, 2023.

(51) Int. Cl. A61L 26/00; B01J 13/00; G01N 33/48

(52) U.S. Cl. CPC A61L 26/0061; A61L 26/0038; B01J 13/0091; A61L 2300/412; A61L 2400/04; A61L 2400/12; G01N 33/48

(57) ABSTRACT

Hybrid aerogels comprising nanofibers and microfibers are provided as well as methods of use thereof and methods of making.

This application claims priority under 35 U.S.C. § 119 (e) to U.S. Provisional Patent Application No. 63/464,732, filed May 8, 2023. The foregoing application is incorporated by reference herein.

This invention was made with government support under Grant No. R01 GM138552 awarded by the National Institutes of Health and Grant No. W81XWH-20-1-0207 awarded by the Department of Defense. The government has certain rights in the invention.

### FIELD OF THE INVENTION

This application relates to the field of aerogels. More specifically, this invention provides nanofiber and microfiber hybrid aerogels, methods of synthesizing, and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Aerogels are an innovative class of micro- or nanostructured materials that offer numerous potential biomedical applications, including regenerative medicine, tissue engineering, wound healing, hemostasis, biosensing, wearable electronics, and drug delivery (Yang, et al. (2017) ACS Nano 11:6817-6824; Cheng, et al. (2022) Nat. Commun., 13:2637; Si, et al. (2014) Nat. Commun., 5:5802; He, et al. (2022) Nat. Commun., 13:4242; Yang, et al. (2019) Small 15:1902826; Li, et al. (2020) Adv. Funct. Mater., 30:2005531; John, et al. (2021) Adv. Healthc. Mater., 10:2100238; John, et al. (2023) Adv. Funct. Mater., 33:2206936; García-González, et al. (2021) J. Controlled Release, 332:40-63; Huang, et al. (2022) Advanced Science, 9:2204519; Gao, et al. (2021) VIEW 2:20200124; Xie, et al. (2021) Adv. Healthc. Mater., 10:2100918; Liu, et al. (2022) Adv. Healthc. Mater., 11:2200499; Wang, et al. (2019) Nano Lett., 19:9112-9120; Weng, et al. (2018) Adv. Healthc. Mater., 7:1701415). In the field of tissue regeneration, aerogels are particularly appealing due to their ultra-high porosity and their lightweight and flexible design. Nevertheless, there are several significant drawbacks to current aerogels that hinder their applications in regenerative medicine. One drawback is their inferior mechanical properties, another is their poor cell infiltration, and a third is their slow and partial shape-recovery. Aerogels often lack structural resilience and flexibility, and thus their use as scaffolds is limited to largely immobile, non-loadbearing tissues (Jiang, et al. (2019) Nat. Commun., 10:3491). Increasing initial concentrations and crosslinking densities or optimization of the crosslinking agent, time and temperature during the fabrication process could enhance mechanical properties but at the expense of porosity, pore interconnectivity, and flexibility (Li, et al. (2020) Adv. Funct. Mater., 30:2005531; John, et al. (2021) Adv. Healthc. Mater., 10:2100238; Zhang, et al. (2021) RSC Adv., 11:10827-10835; Khedaioui, et al. (2019) Angewandte Chemie Intl. Ed., 58:15883-15889; Wang, et al. (2020) Chem. Engr. J., 399:125698; He, et al. (2019) Chem. Engr. J., 371:34-42; Wang, ct al. (2020) Chem. Engr. J., 399:125698; Zhou, et al. (2020) J. Appl. Mech., 87:031002). This mechanical strength-flexibility conflict limits the use of aerogels in wide applications.

To promote cellular infiltration, patterned macrochannels were created within nanofiber aerogels using 3D printed sacrificial templates (John, et al. (2023) Adv. Funct. Mater., 33:2206936; Du, et al. (2021) Nat. Commun., 12:4733). However, this approach would increase the complexity and cost of fabrication and the mechanical properties of obtained aerogels were still not improved.

Different materials—such as polyimide (Li, et al. (2021) ACS Nano 15:4759-4768; Hou, et al. (2021) Chem. Eng. J., 417:129341), hydroxyapatite (Zheng, et al. (2022) Chem. Eng. J., 430:132912), polyurethane (Zhou, et al. (2016) Chem. Engr. J., 302:155-162; Malakooti, et al. (2021) ACS Appl. Polym. Mater., 3:5727-5738), gelatin (John, et al. (2023) Adv. Funct. Mater., 33:2206936; Jiang, et al. (2019) Chem. Engr. J., 358:1539-1551), cellulose (Heise, et al. (2021) Adv. Mater., 33:2004349; Song, et al. (2018) ACS Nano, 12:140-147), and chitin (Liu, et al. (2019) ACS Nano 13:2927-2935)—have been used for fabrication of aerogels with three-dimensional (3D), fibrillar network structures, which helps address the issues of slow and partial shape-recovery though the improvement in mechanical properties is minimal. In addition, these aerogels often exhibit non-interconnected pores, small pore size, and/or poor mechanical properties (compression modulus: 10-2 to 101 MPa) (He, et al. (2022) Nat. Commun., 13:4242; Du, et al. (2021) Nat. Commun., 12:4733; Wang, et al. (2022) Advanced Materials 34:2108325).

For at least the foregoing reasons, developing aerogels with high strength and flexibility for fast full shape recovery that maintain high porosity and pore interconnectivity while promoting cellular infiltration remains a significant challenge.

### SUMMARY OF THE INVENTION

In accordance with the instant invention, compositions comprising an intertwined network of nanofibers and microfibers are provided. The compositions are generally referred to as aerogels herein. In certain embodiments, the aerogels further comprise a binder (e.g., gelatin). In certain embodiments, the nanofibers and microfibers are coated with the binder. In certain embodiments, the nanofibers and microfibers and the binder are crosslinked (e.g., by a chemical crosslinker such as glutaraldehyde). In certain embodiments, the average diameter of the microfibers is about 5 to about 200 times, particularly about 10 to about 100 times, greater than the average diameter of the nanofibers. In certain embodiments, the weight ratio of nanofibers to microfibers is from about 25:75 to about 75:25. In certain embodiments, the nanofibers have an average diameter from about 100 nm to about 500 nm. In certain embodiments, the microfibers have an average diameter from about 1 μm to about 30 μm. In certain embodiments, the nanofibers have an average length of less than about 250 μm. In certain embodiments, the microfibers have an average length less than about 10 mm. In certain embodiments, the nanofibers and microfibers are polymeric. In certain embodiments, the nanofibers and microfibers comprise the same polymer. In certain embodiments, the nanofibers and microfibers comprise different polymers. In certain embodiments, the nanofibers and/or microfibers comprise hydrophobic polymers. In certain embodiments, the nanofibers and microfibers comprise polycaprolactone (PCL). In certain embodiments, the nanofibers and/or microfibers further comprise a poloxamer. In certain embodiments, the nanofibers and/or microfibers are electrospun. In certain embodiments, the nanofibers and/or microfibers are wet spun. In certain embodiments, the aerogels further comprise a drug, bioactive agent, polypyrrole, magnetic nanoparticle, cells and/or tissue.

In accordance with another aspect, methods of synthesizing the aerogels of the instant invention are provided. In certain embodiments, the method comprises freczing, freeze drying, and/or freeze casting a composition comprising nanofibers and microfibers.

In accordance with another aspect of the instant invention, methods of treating, inhibiting, and/or preventing a disease or disorder are provided. Methods of treating an injury or wound in a subject are also provided. Methods of inhibiting and or treating bleeding and/or hemorrhaging in a subject are provided. Methods of regenerating tissue in a subject are provided. Generally, the methods comprise delivering, administering, or contacting a desired site (e.g., site of action) in the subject with an aerogel of the instant invention. The aerogels of the instant invention may be administered by any means including by injection (e.g., via a syringe, cannula, needle, etc.). In certain embodiments, the wound or injury is a skin injury, skin lesion, or diabetic wound.

In accordance with another aspect of the instant invention, methods of collecting a biological sample are provided. In certain embodiments, the method comprises contacting a site on or in a subject with an aerogel of the instant invention. In certain embodiments, the aerogel is compressed upon contacting the site and/or subject and allowed to expend while still in contact with the site and/or subject.

### BRIEF DESCRIPTION OF THE DRAWINGS

FIG. 1A provides images of 3D hybrid aerogels prepared with 50:50 w/w ratio of polycaprolactone (PCL) nanofiber (NF) and microfiber (MF) (NF/MF-A1). FIG. 1B provides images of NF/MF-A1 aerogels supported by a wooden wall or powder-free hand glove against gravity due to electrostatic forces. FIGS. 1C and 1D provide graphs of the density and porosity, respectively, of samples. n=5, *p<0.01, ****p<0.0001, ns=a non-significant value of >0.05. FIG. 1E-1I provide SEM images of NFA, MFA, NF/MF-A1, NF/MF-A2, and NF/MF-A3, respectively. Right panels are enlarged images of rectangular areas in the left panels. Inset in FIG. 1G is an enlarged image of the rectangular area in the right panel of FIG. 1G. NFA: nanofiber aerogels. MFA: microfiber aerogels. NF/MF-A1: hybrid aerogels containing NF/MF at a ratio of 50:50 (w/w). NF/MF-A2: hybrid aerogels containing NF/MF at a ratio of 75:25 (w/w). NF/MF-A3: hybrid aerogels containing NF/MF at a ratio of 25:75 (w/w). FIGS. 1J and 1K provide graphs of the distribution of diameter and length, respectively, of short NFs. FIGS. 1L and 1M provide graphs of the distribution of diameter and length, respectively, of short MFs. FIG. 1N provides images showing hybrid aerogels (NF/MF-A1) with various customized shapes. Scale bar=1 cm. FIGS. 1O-1Q provide statistical histograms of fiber orientations in NF/MF-A1, NF/MF-A2, and NF/MF-A3, respectively. FIG. IR provides a graph of the pore size measurements of samples. Data are presented as mean values±SD, N=5. The significant difference was detected by oneway ANOVA with Tukey's multiple comparisons test. The ‘ns’ indicated no significant difference, *p<0.01, ****p<0.0001.

FIG. 2A provides images showing the compression and bending resistance capability of cylindrical hybrid aerogels (NF/MF-A1). FIG. 2B provides compression stress-strain curves of NFA, MFA, NF/MF-A1, NF/MF-A2, and NF/MF-A3 at 90% strain. FIGS. 2C and 2D provide graphs of the maximum compressive strength and Young's modulus, respectively, at 50, 70, and 90% compressive strains. The p-values are given in Tables 1-2. FIG. 2E-2G provide graphs of the changes in length of NFA, MFA, and NF/MF-A1, respectively, as a function of their ultra-resilient properties during cyclic compression-relaxation test at 90% compressive strain. FIGS. 2H-2I provide graphs of maximum tearing strength and fracture energies, respectively. n=5, *p<0.05, **p<0.01, ***p<0.001, ****p<0.0001, ns=a non-significant value of >0.05. FIG. 2J provides a graph of the comparison of mechanical properties of hybrid aerogels with other polymeric aerogels known for high mechanical properties. (1) NFA; (2) MFA; (3) NF/MF-A1; (4) NF/MF-A2; (5) NF/MF-A3; (6) composite nanofiber aerogel with porosity of 91% (CNA-91); (7) composite nanofiber aerogel with porosity of 87% (CNA-87); (8) composite nanofiber aerogel with porosity of 83% (CNA-83); (9) composite nanofiber aerogel with porosity of 76% (CNA-76); (10) bacterial cellulose (BC); (11) BC/poly(3,4-theylenedioxythiophene)/single walled carbon nanotube (BC/PEDOT/SWNT); (12) chitin, (13) chitosan, (14) polyimide (PI); (15) PI/carbon nanotube (PI/CNT); and (16) PI/graphene. FIG. 2K provides a graph of percentages of shape recovery of tested samples in pH 2.0 after compressing the aerogels in vertical directions. FIG. 2L provides a graph of the percentages of shape recovery in PBS after compressing the aerogels in horizontal and vertical directions. FIG. 2M provides a graph of shape recovery time. Data are presented as mean±SD, n=5. GS: gelatin sponge. FIGS. 2N and 2O provide graphs of the changes in length of NF/MF-A2 and NF/MF-A3, respectively during 10 cycles of compression-relaxation test at 90% compressive strain. FIG. 2P provides schematics which illustrate the fluid responsive shape-memory characteristics of hybrid aerogels. FIG. 2Q provides images showing the experimental setup for each cycle. FIGS. 2R-2V provide graphs of the results from the cyclic compression test of NFA, MFA, NF/MF-A1, NF/MF-A2, and NF/MF-A3, respectively, under 50%, 70%, and 90% compressive strains. FIG. 2W provide images of shape recoverable heart- and breast-shaped hybrid aerogels. Scale=1 cm. FIGS. 2X and 2Y provide SEM images showing the microstructure of the hybrid aerogels (NF/MF-A1) after compression and shape recovery, respectively. FIG. 2Z provides a schematic of a proposed mechanism of high resilience of NF/MF-A1 hybrid aerogels under 90% compressive strain.

FIGS. 3A and 3B provide confocal laser scanning microscopy (CLSM) images showing 3D and orthogonal view of GFP-labeled dermal fibroblasts in the aerogels after seeding for 3 and 7 days. FIG. 3C provides CLSM images showing human neural progenitor/stem cells cultured on NFA, MFA, and NF/MF-A1 samples for 7 days. Neurons were stained with β-III. 3D view and depth analysis reveal the distribution of neurons/neurites on NFA, MFA, and NF/MF-A1 after 7 days of culture. FIG. 3D provides a schematic illustrating the migration of cells from bottom to top. FIG. 3E provides CLSM images showing GFP-labeled dermal fibroblasts on the bottom surface and top surface of NF/MF-A1 aerogels at day 0, 3, 6, and 9. Scale bar=100 μm for FIGS. 3A-3C; 1 mm for FIG. 3E. FIGS. 3F-3H provide CLSM images showing live/dead staining of HaCaT cells cultured on NFA, MFA, and NF/MF-A1 cylindrical aerogels, respectively, for 36 hours (diameter=8 mm, height=2 mm). Scale bar=100 μm.

FIG. 31 provides a graph of migration of neural stem cells and axonal growth in the aerogels. n=3, ****p<0.0001, ns=a non-significant value of >0.05. FIG. 3J provides a graph of the migration of GFP-labeled dermal fibroblasts from the bottom surface to top surface of NF/MF-A1 at day 0, 3, 6, and 9. FIG. 3K provides confocal images showing GFP-labeled dermal fibroblasts in the cross-sectional area of NF/MF-A1 before and after injection. Scale bar=100 μm. FIG. 3L provides a graph of the percentages of viable cells present within NF/MF-A1 before and after injections. Data are presented as mean±SD, n=5.

FIG. 4A provides a schematic illustrating different cross-sections of each explant. FIG. 4B provides H & E staining images of native subcutaneous tissue.

FIGS. 4C and 4D provide H & E staining images of different cross-sections (sections 1-5, left to right) of each explant at day 14 and 28, respectively. Circles indicate the newly formed blood vessels inside aerogels. Scale bar=50 μm. FIG. 4E provides a graph of the cell counts per region of interest (ROI) in each explant (left to right: 2D NF mat; NFA; MFA; and NF/MF-A1). FIG. 4F provides a graph of percentages of cell infiltrated area in each explant (left to right: 2D NF mat; NFA; MFA; and NF/MF-A1). FIG. 4G provides a graph of the number of newly formed blood vessels in each explant (left to right: 2D NF mat; NFA; MFA; and NF/MF-A1). n=12, *p<0.05, **p<0.01, ***p<0.001, ****p<0.0001.

FIG. 5A provides images of trichrome staining of the native subcutaneous tissue. FIGS. 5B and 5C provides images of trichrome staining of different cross-sections of explants after implantation for 14 and 28 days, respectively (sections 1-5, left to right). Deposited collagen areas are visible. FIG. 5D provides a graph of the percentages of the deposited collagen area in each explant (left to right: 2D NF mat; NFA; MFA; and NF/MF-A1). FIG. 5E provides a graph of the quantification of the deposited collagen in each explant (left to right: 2D NF mat; NFA; MFA; and NF/MF-A1). n=12, *p<0.05, **p<0.01, ***p<0.001, ****p<0.0001. Scale bar=50 μm. FIG. 5F provides a schematic illustrating the fabrication of electrically conductive aerogels. FIG. 5G provides a Schematic illustrating the effect of compressive strain on the electrical resistance (left panel) and the related equations (right panel). R₀ and R represent the incipient resistance and momentary resistance. H₀ and H represent the original height and the height after compression. FIG. 5H provides a graph of the change in resistance at different compressive strains. FIG. 5I provides a graph of the cyclic change in electrical resistance at 25% and 90% compressive strains. FIG. 5J provides a schematic illustrating the resistance value of conductive hybrid aerogels changes according to the different degrees of compression. FIG. 5K provides a schematic illustrating the fabrication of hybrid aerogels with incorporation of Fe₃O₄ nanoparticles. FIG. 5L provides images showing NF/MF-A1 (left) fails to respond to magnetic field and the hybrid aerogel (right) after incorporation of Fe₃O₄ nanoparticles is responsive to a magnetic field.

FIG. 6A provides a schematic showing the short nanofibers and microfibers fabrication processes, the fabrication steps involved in making hybrid aerogels, and the utilization of hybrid aerogels in regenerative wound healing in genetically engineered diabetic mice (DB/DB mice). FIG. 6B provides images of the post-treatment progression of diabetic wounds in DB/DB mice at intervals of 7-, 14-, and 21-days post-surgery and the indicated treatment. Control: Mice which received no treatment after surgery. FIG. 6C provides images of H&E stained images depicting the histological analysis of healed wounds at days 7, 14, and 21 after the indicated treatments. FIG. 6D provides a graph of the percentage of wound healing observed in in vivo studies conducted at 7, 14, and 21 days. ▪ Control; ● NFA; ▴MFA; ▾NF/MF-A. A p-value greater than 0.05 indicates non-significance (ns); ****p-value<0.0001. FIG. 6E provides images of MTC staining results of healed wounds at days 7, 14, and 21 after the indicated treatments.

### DETAILED DESCRIPTION OF THE INVENTION

Aerogels show promise as materials for soft tissue engineering due to their light weight, high porosities, and design flexibility. However, most aerogels have poor mechanical properties such as brittleness and low elastic moduli. While increasing crosslinking density may improve mechanics, it also imparts brittleness. In soft tissue engineering, the ability to withstand mechanical loads from mobile tissue is critical. To engineer aerogels that are both mechanically resilient and optimal for tissue regeneration, a hybrid aerogel comprising self-reinforcing networks of micro- and nanofibers is provided herein. Nanofiber segments physically entangle microfiber pillars, allowing the structures to transfer stress effectively through the intertwined fiber networks. Hybrid aerogels of the instant invention have high specific tensile moduli (e.g., ˜1961.3 MPa cm³ g⁻¹) and fracture energies (e.g., ˜7408.1 J m⁻²), as well as exhibit super-elastic properties with rapid shape recovery (e.g., ˜1.8 seconds). These aerogels induce rapid tissue ingrowth, extracellular matrix deposition, and neovascularization after subcutaneous implants to rats. They can be delivered via minimally invasive procedures for engineering soft tissues. Furthermore, functionalization can tailor hybrid aerogels to desired properties, such as magnetically responsive or electrically conductive for specific applications, including pressure sensing and actuation.

As stated above, the novel aerogels of the instant invention comprise both polymeric nanofibers and microfibers. Without being bound by theory, soft nanofibers form a network that intertwines with a harder microfiber network. During compression, both networks undergo deformation under external stress without breaking because the aerogel allows mechanical stress to be transmitted through the entangled fibrillar network, ensuring shape recovery and preventing mechanical failure (Ducrot, et al. (2014) Science 344:186-189; Sun, et al. (2012) Nature 489:133-136). The entanglement of fibrillar segments functions as additional binders that cannot be disassembled without breakage in this hybrid aerogel containing dual-scale sizes (Edwards, S. F. (1967) Proc. Phys. Soc., 91:513-519; Gong, et al. (2003) Advanced Materials 15:1155-1158). This system has at least the following advantages. The low crosslinking density and fibrillar networks allow the aerogels to be highly flexible and elastic. The multiscale fibrillar networks act as mechanical supports, imparting stiffness and rigidity, while nanofibers provide a biomimetic morphology, and microfibers assist in forming large pores for cell infiltration.

The hybrid aerogels of the instant invention comprise short nanofibers and microfibers that are intertwined and can be coated with a binder. Crosslinking of the binder reinforces the self-entangled, multiscale fibrillar network structures. Several important findings are demonstrated including, without limitation: (i) crosslinking and entanglement between nanofibers and microfibers enhance the mechanical strength and flexibility of hybrid aerogels; (ii) the hybrid aerogels act as resilient substrate during compressing by storing a large amount of elastic energy, thus preventing mechanical failure under high mechanical loads; (iii) the presence of dual-scale sized fibers is crucial for flexibility and shape-recovery, which allows for the delivery of hybrid aerogels via a cannula or catheter; (iv) the hybrid aerogels promote tissue ingrowth, extracellular matrix (ECM) production, and angiogenesis. The aerogels possess suitable porosity (e.g., above, 80%, above 85%, above 90%, or greater) for cellular infiltration. Further, it has been demonstrated that compressed aerogels can re-expand in blood within 5 seconds (e.g., 2 seconds) and that aerogels (50:50) have a similar blood absorption capacity and blood clotting time to XSTAT® (RevMedx, Wilsonville, OR) with similar adhesion to red blood cells, platelets, and blood proteins.

In accordance with the instant invention, composites comprising nanofibers and microfibers are provided. Methods of making and methods of using the composites are also provided. Generally, the composites of the instant invention are referred to herein as aerogels. Aerogels are a network of interconnected structures comprising a corresponding network of interconnected pores. Generally, aerogels are light and porous solid foams. In certain embodiments, the aerogels of the instant invention comprise polymeric nanofibers and microfibers, particularly short polymeric nanofibers and microfibers. The aerogels of the instant invention may be molded into any three-dimensional shape. The aerogels of the instant invention can be used, for example, in the treatment and/or prevention of bleeding/hemorrhaging, wound healing, and/or tissue regeneration.

As used herein, nanofibers are fibers having a diameter less than about 1 μm (e.g., average diameter), but greater than about 1 nm. In certain embodiments, the nanofibers of the aerogel have a diameter (e.g., average diameter) of about 1 nm to about 900 nm, about 1 nm to about 750 nm, about 1 nm to about 500 nm, about 100 nm to about 750 nm, about 100 nm to about 600 nm, about 100 nm to about 500 nm, about 200 nm to about 500 nm, about 250 nm to about 500 nm, about 250 nm to about 450 nm, about 300 nm to about 400 nm, or about 325 nm to about 375 nm. In certain embodiments, the nanofibers of the aerogel have a diameter (e.g., average diameter) of at least about 1 nm, at least about 10 nm, at least about 20 nm, at least about 50 nm, at least about 100 nm, at least about 150 nm, at least about 200 nm, at least about 250 nm, or at least about 300 nm. In certain embodiments, the nanofibers of the aerogel have a diameter (e.g., average diameter) of less than about 900 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, or less than about 250 nm.

In certain embodiments, the nanofibers are electrospun nanofibers. In certain embodiments, the nanofibers of the aerogel are short segments. Segments of nanofibers can be fabricated by any method. In certain embodiments, the nanofiber segments are derived from longer nanofibers (e.g., electrospun nanofibers), such as by cutting (e.g., cryocutting) and/or homogenization (e.g., by sonication, particularly probe sonication). In certain embodiments, the nanofibers are less than about 500 μm in length, less than about 40 μm in length, less than about 300 μm in length, less than about 250 μm in length, less than about 200 μm in length, less than about 150 μm in length, or less than about 100 μm in length. In certain embodiments, the nanofibers are at least about 1 μm in length, at least about 5 μm in length, at least about 10 μm in length, at least about 15 μm in length, at least about 20 μm in length, at least about 25 μm in length, at least about 30 μm in length, at least about 35 μm in length, at least about 40 μm in length, at least about 45 μm in length, or at least about 50 μm in length. In certain embodiments, the length (e.g., median or mean length) of the nanofibers is about 1 μm to about 500 μm in length, about 10 μm to about 250 μm in length, about 20 μm to about 200 μm in length, about 30 μm to about 175 μm in length, about 50 μm to about 150 μm in length, about 65 μm to about 125 μm in length, or about 70 μm to about 110 μm in length.

Generally, microfibers are fibers having a diameter greater than about 1 μm (e.g., average diameter), but less than about 1 millimeter. In certain embodiments, the microfibers of the aerogels of the instant invention may have a diameter (e.g., average diameter) as short as 0.5 μm, but will generally be at least 1 μm in diameter. In certain embodiments, the microfibers of the aerogel have a diameter (e.g., average diameter) of about 1 μm to about 500 μm, about 1 μm to about 400 μm, about 1 μm to about 300 μm, about 1 μm to about 250 μm, about 1 μm to about 200 μm, about 1 μm to about 150 μm, about 1 μm to about 100 μm, about 1 μm to about 50 μm, about 1 μm to about 30 μm, about 5 μm to about 25 μm, or about 10 μm to about 20 μm. In certain embodiments, the microfibers of the aerogel have a diameter (e.g., average diameter) of at least about 0.5 μm, at least about 1 μm, at least about 2.5 μm, at least about 5 μm, at least about 7.5 μm, at least about 10 μm, at least about 12.5 μm, or at least about 15 μm. In certain embodiments, the microfibers of the aerogel have a diameter (e.g., average diameter) of less than about 500 μm, less than about 400 μm, less than about 300 μm, less than about 200 μm, less than about 100 μm, less than about 75 μm, less than about 50 μm, less than about 40 μm, less than about 30 μm, less than about 20 μm, or less than about 15 μm.

In certain embodiments, the microfibers of the aerogel are short segments. Segments of microfibers can be fabricated by any method. In certain embodiments, the microfiber segments are derived from longer microfibers (e.g., wet spun microfibers), such as by cutting (e.g., cryocutting) and/or homogenization (e.g., by sonication, particularly probe sonication). In certain embodiments, the microfibers are less than about 100 mm in length, less than about 50 mm in length, less than about 25 mm in length, less than about 20 mm in length, less than about 15 mm in length, less than about 10 mm in length, less than about 7.5 mm in length, less than about 5 mm in length, less than about 4 mm in length, less than about 3 mm in length, less than about 2 mm in length, or less than about 1 mm in length. In certain embodiments, the microfibers are at least about 0.5 mm in length, at least about 1 mm in length, at least about 1.5 mm in length, or at least about 2 mm in length. In certain embodiments, the length (e.g., median or mean length) of the microfibers is about 0.1 mm to about 100 mm in length, about 0.1 mm to about 50 mm in length, about 0.1 mm to about 25 mm in length, about 0.5 mm to about 25 mm in length, about 0.5 mm to about 20 mm in length, about 1 mm to about 20 mm in length, about 0.5 mm to about 15 mm in length, about 1 mm to about 15 mm in length, about 0.5 mm to about 10 mm in length, about 1 mm to about 10 mm in length, about 0.5 mm to about 5 mm in length, about 1 mm to about 5 mm in length, about 0.5 mm to about 3 mm in length, or about 1 mm to about 3 mm in length.

In certain embodiments, the diameter (e.g., average diameter) of the microfibers of the aerogel is about 5 to about 200 times, about 10 to about 100 times, about 15 to about 85 times, about 25 to about 75 times, about 35 to about 60 times, or about 40 to about 50 times greater than the diameter (e.g., average diameter) of the nanofibers of the aerogel.

As explained herein, the aerogels of the instant invention comprise nanofibers and microfibers. In certain embodiments, the aerogel has from 1% to 99% nanofibers and 1% to 99% microfibers. In certain embodiments, the aerogel comprises 75% nanofibers and 25% microfibers, 50% nanofibers and 50% microfibers, or 25% nanofibers and 75% microfibers. In certain embodiments, the ratio (e.g., by weight) of nanofibers to microfibers is about 1:99 to about 99:1, about 5:95 to about 95:5, about 10:90 to about 90:10, about 15:85 to about 85:15, about 20:80 to about 80:20, about 25:75 to about 75:25, about 30:70 to about 70:30, about 35:65 to about 65:35, about 40:60 to about 60:40, about 45:55 to about 55:45, or about 50:50. In certain embodiments, the ratio (e.g., by weight) of nanofibers to microfibers is from about 25:75 to about 75:25. In certain embodiments, the ratio (e.g., by weight) of nanofibers to microfibers is about 75:25, about 50:50, or about 25:75.

The nanofibers and microfibers of the instant invention may be comprised of any polymer (e.g., polymer fibers). In certain embodiments, the polymer is biocompatible and/or biodegradable. In certain embodiments, the polymer is hydrophobic, hydrophilic, or amphiphilic. In certain embodiments, the polymer is hydrophobic. In certain embodiments, the nanofibers and/or microfibers comprise electrospun fibers. In certain embodiments, the nanofibers and/or microfibers comprise wet spun fibers. In certain embodiments, the nanofibers and microfibers comprise the same polymer. In certain embodiments, the nanofibers comprise a different polymer than the microfibers. In certain embodiments, the nanofibers comprise a first polymer(s) and the microfibers comprise a second polymer(s). In certain embodiments, the first polymer(s) are the same as the second polymer(s). In certain embodiments, the first polymer(s) are different than the second polymer(s).

The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene). In a particular embodiment, the hydrophobic polymer comprises polycaprolactone (PCL).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, clastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment or block) and a hydrophobic polymer (e.g., segment or block)—such as those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

In certain embodiments, the fiber comprises polymethacrylate, poly vinyl phenol, polyvinylchloride, cellulose, polyvinyl alcohol, polyacrylamide, poly(lactic-co-glycolic acid) (PLGA), collagen, polycaprolactone, polyurethanes, polyvinyl fluoride, polyamide, silk, nylon, polybenzimidazole, polycarbonate, polyacrylonitrile, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, starch-acrylonitrile co-polymers, bioactive glass, and/or combinations of two or more polymers. Multiple polymers may be mixed to form the fibers. The polymers may be mixed in equal ratios or various ratios depending on the desired properties of the nanofibers.

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in nanofibers, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium alginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO3, PCL/HA, PLLA/HA, gelatin/HA, PCL/collagen/HA, collagen/HA, gelatin/siloxane, PLLA/MWNTs/HA, PLGA/HA).

In certain embodiments, the nanofiber and/or microfiber further comprises at least one amphiphilic block copolymer comprising hydrophilic poly(ethylene oxide) (PEO) and hydrophobic poly(propylene oxide) (PPO). In certain embodiments, the nanofiber and/or microfiber comprises a poloxamer (e.g., Pluronic®) or an amphiphilic triblock copolymer comprising a central hydrophobic PPO block flanked by two hydrophilic PEO blocks (i.e., an A-B-A triblock structure). In certain embodiments, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In certain embodiments, the nanofiber and/or microfiber comprises poloxamer 188. In certain embodiments, the nanofiber and/or microfiber comprises poloxamer 407 (Pluronic® F127). The amphiphilic block copolymer (e.g., poloxamer) may be added in various amounts to the polymer solution during the synthesis process (e.g., electrospinning). In certain embodiments, about 0% to about 20%, about 0% to about 15%, about 0% to about 10%, about 0.1% to about 5%, about 0.5% to about 2%, or about 0.1% to about 1.0% (e.g., w/v) of the polymer solution is an amphiphilic block copolymer (e.g., a poloxamer (e.g., poloxamer 407 and/or poloxamer 188)). In certain embodiments, about 0.1% to about 50%, about 0.1% to about 40%, about 0.1% to about 30%, about 0.1% to about 25%, about 0.1% to about 20% (e.g., w/v) of the polymer solution is polymer (e.g., PCL).

In certain embodiments, the polymer solution comprises about 10% polymer (w/v) (e.g., PCL) and about 0.5% poloxamer 407 (w/v) (Pluronic® F127). In certain embodiments, the polymer solution comprises PCL and poloxamer 407 in a ratio (e.g., by weight) of about 200:1 to about 2:1, about 100:1 to about 4:1, about 50:1 to about 8:1, about 40:1 to about 10:1, about 30:1 to about 15:1, or about 20:1.

In certain embodiments, the nanofiber and/or microfiber comprises PCL. In certain embodiments, the nanofiber comprises PCL. In certain embodiments, the microfiber comprises PCL. In certain embodiments, the nanofiber and/or microfiber comprises PCL and poloxamer 407. In certain embodiments, the nanofiber comprises PCL and poloxamer 407 and the microfiber comprises PCL. In certain embodiments, the nanofiber and/or microfiber comprises PGLA (e.g. PGLA 90:10).

In certain embodiments, the nanofiber and/or microfiber of the aerogel are plasma treated (e.g., air plasma or oxygen plasma). Generally, plasma treatment generates negatively charged groups (e.g., carboxyl groups) that enhance the interaction between the fiber and other agents.

In certain embodiments, the nanofiber and/or microfiber of the aerogel further comprise (e.g., are coated) with a coating material such as gelatin. Generally, the coating material is a binder. While gelatin is described herein as the coating material, other coating materials (e.g., binder) may be used (e.g., coating materials with adhesive properties and/or water absorbing properties). For example, the nanofiber and/or microfiber may comprise or be coated with a hydrogel, collagen, a proteoglycans, elastin, a glycosaminoglycan (e.g., hyaluronic acid, heparin, chondroitin sulfate, or keratan sulfate), gelatin, gelatin methacryloyl (GelMA), agarose, alginate, sodium alginate, chitosan, chitin, starch, pectin, cellulose, methylcellulose, poly(organophosphazenes), fibrinogen, fibronectin, laminin, poly(N-isopropylacrylamide), poly(vinyl methyl ether), poly(ethylene glycol), poly(propylene glycol), poly(methacrylic acid), poly(vinyl alcohol), sodium polyacrylate, starch-acrylonitrile co-polymers, a glue (bioadhesive) (e.g., fibrin glue), and/or other natural or synthetic hydrogels, and derivatives thereof (e.g., del Valle et al., Gels (2017) 3:27). In certain embodiments, the nanofiber and/or microfiber may comprise or be coated with gelatin, collagen, GelMA, agarose, poly(organophosphazenes), sodium alginate, hyaluronic acid, fibrinogen, fibronectin, laminin, poly(N-isopropylacrylamide), poly(vinyl methyl ether), poly(ethylene glycol), poly(propylene glycol), poly(methacrylic acid), poly(vinyl alcohol), chitosan, starch, pectin, cellulose, methylcellulose, sodium polyacrylate, or starch-acrylonitrile co-polymers. As used herein, a hydrogel is a polymer matrix able to retain water, particularly large amounts of water, in a swollen state. In certain embodiments, the coating material is gelatin, chitosan, collagen, cellulose, chitin, a hydrogel, or a glue (bioadhesive) (e.g., fibrin glue). In certain embodiments, the coating material is gelatin.

The term “coat” refers to a layer of a substance/material on the surface of an aerogel and/or the fibers of the aerogel. In certain embodiments, the coating covers the surface of the nanofibers and/or microfibers. Coatings may impregnate the aerogel (e.g., form a layer within pores). Further, while a coating may cover 100% of the aerogel, a coating may also cover less than 100% of the surface of the aerogel (e.g., at least about 50%, at least about 75%, at least about 80%, at least about 85%, at least about 90%, at least about 95%, at least about 98%, or more of the surface may be coated).

In certain embodiments, the aerogel is crosslinked. In certain embodiments, the nanofiber and/or microfiber of the aerogel are crosslinked. In certain embodiments, the nanofiber and/or microfiber of the aerogel are crosslinked after application of the coating material (e.g., binder) (e.g., the coating material (e.g., binder) is crosslinked). Crosslinking may be done using a variety of techniques including thermal crosslinking, chemical crosslinking, UV-crosslinking, and photo-crosslinking. For example, the nanofiber and/or microfiber of the aerogel of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In certain embodiments, the crosslinker is glutaraldehyde (e.g., glutaraldehyde vapor).

In certain embodiments, the aerogel further comprises a drug and/or bioactive agent. In certain embodiments, the drug or bioactive agent to helps treat, prevent, or modulate biological processes including but not limited to enhance blood clotting (such as clotting proteins), enhance wound healing and/or enhance tissue regeneration (such as growth factors), treatment/prevention of infections (antimicrobials such as antibacterials, antivirals and antifungals), treat and/or control pain (such as analgesics or other pain medicines), treat or prevent damage from chemical, biological, radiological and nuclear mitigants, and control of inflammation (using small molecules, peptides and other agents capable of modulating inflammation). In certain embodiments, the drugs or bioactive agents is conjugated (e.g., directly or via a linker (e.g., crosslinker)) to the nanofibers and/or microfibers of the aerogel. In certain embodiments, the drug or bioactive agent are packaged or contained within the aerogel (e.g., without direct conjugation to the nanofibers and/or microfibers). In certain embodiments, the drugs or bioactive agents are administered with but not incorporated into the aerogel.

Bioactive agents include but are not limited to small molecules, proteins, peptides, antibodies, antibody fragments, nucleic acid, DNA, RNA, and other known biologic substances, particularly those that have therapeutic use. In certain embodiments, the agent is an antibiotic, analgesic, growth factor, cytokine, or clotting factors. In certain embodiments, the agent is a drug or therapeutic agent (e.g., a small molecule) (e.g., analgesic, growth factor, anti-inflammatory, signaling molecule, growth factor, cytokine, antimicrobial (e.g., antibacterial, antibiotic, antiviral, and/or antifungal), hormone (e.g., insulin), ephrins, hemostatic agent (e.g., blood clotting agent, factor, or protein), pain medications (e.g., anesthetics), etc.). In certain embodiments, the agent enhances tissue regeneration, tissue growth, and wound healing (e.g., growth factors). In certain embodiments, the agent treats/prevents infections (e.g., antimicrobials such as antibacterials, antibiotics, antivirals and/or antifungals). In certain embodiments, the agent is an antimicrobial, particularly an antibacterial. In certain embodiments, the agent enhances wound healing and/or enhances tissue regeneration (e.g., bone, tendon, cartilage, skin, nerve, and/or blood vessel). Such agents include, for example, growth factors, cytokines, chemokines, immunomodulating compounds, and small molecules. Growth factors include, without limitation: platelet derived growth factors (PDGF), vascular endothelial growth factors (VEGF), epidermal growth factors (EGF), neuregulins, fibroblast growth factors (FGF; e.g., basic fibroblast growth factor (bFGF)), insulin-like growth factors (IGF-1 and/or IGF-2), bone morphogenetic proteins (e.g., BMP-2, BMP-7, BMP-12, BMP-9; particularly BMP-2 fragments, peptides, and/or analogs thereof), transforming growth factors (e.g., TGFα, TGFβ, TGFβ3), tumour Necrosis Factor alpha (TNF alpha), nerve growth factors (NGF), neurotrophic factors, stromal derived factor-1 (SDF-1), granulocyte-macrophage colony-stimulating factor (GM-CSF), macrophage colony-stimulating factor (M-CSF), granulocyte-colony stimulating factor (G-CSF), erythropoietin (EPO), glial cell-derived neurotrophic factors (GDNF), hepatocyte growth factors (HGF), keratinocyte growth factors (KGF), neurotrophin, and/or growth factor mimicking peptides (e.g., VEGF mimicking peptides). In certain embodiments, the growth factor is bFGF. Chemokines include, without limitation: CCL21, CCL22, CCL2, CCL3, CCL5, CCL7, CCL8, CCL13, CCL17, CXCL9, CXCL10, and CXCL11. Cytokines include without limitation IL-2 subfamily cytokines, interferon subfamily cytokines, IL-10 subfamily cytokines, IL-1, I-18, IL-17, tumor necrosis factor, and transforming-growth factor beta superfamily cytokines. Examples of small molecule drugs/therapeutic agents include, without limitation, simvastatin, kartogenin, retinoic acid, paclitaxel, vitamins (e.g., vitamin D3), etc. In certain embodiments, the agent is a blood clotting factor such as thrombin or fibrinogen. In a particular embodiment, the agent is a bone morphogenetic protein (e.g., BMP-2, BMP-7, BMP-12, BMP-9; particularly human; particularly BMP-2 fragments, peptides, and/or analogs thereof

Antimicrobials may include, without limitation, small molecules, peptides, proteins, DNA, RNA, and other known biologic substances. In certain embodiments, the antimicrobial is a small molecule. In certain embodiments, the antimicrobial is an antiviral, antifungal, antibiotic or antibacterial, particularly an antibiotic or antibacterial. Examples of antimicrobials include, without limitation, antibiotics such as beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., crythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linczolid, posizolid, tedizolid, radezolid, contezolid, etc.), lipopeptides (e.g., daptomycin), glycylcyclines (e.g., tigecycline), moenomycins, aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin); silver containing compounds (e.g., silver ions, silver nitrate, silver nanoparticles, colloidal silver, etc.), gallium containing compounds (e.g., gallium ions, gallium nitrate, gallium nanoparticles, colloidal gallium, etc.), and antimicrobial peptides. Examples of antifungals include, without limitation, amphotericin B, pyrimethamine, thiazoles, allylamines, flucytosine, caspofungin acetate, fluconazole, griseofulvin, terbinafine, amorolfine, imidazoles, triazoles (e.g., voriconazole), flutrimazole, cilofungin, cchinocandines, pneumocandin omoconazole terconazole, nystatin, natamycin, griscofulvin, ciclopirox, naftifine, and itraconazole. In a particular embodiment, the antimicrobial is an antibiotic. In certain embodiments, the antimicrobial is an antimicrobial peptide. In a particular embodiment, the aerogel comprises an antimicrobial peptide and at least one other antimicrobial (e.g., antibiotic). Antimicrobial peptides may be therapeutically effective against one or more bacteria. Examples of antimicrobial peptides are provided in the Antimicrobial Peptide Database (aps.unmc.cdu/AP/main.php). Examples of antimicrobial peptides are also disclosed in U.S. Pat. Nos. 7,465,784, 9,580,472, 10,144,767, U.S. Patent Application Publication No. 20090156499, U.S. Patent Application Publication No. 20150259382, U.S. Patent Application Publication No. 20140303069, and PCT/US2019/039792, each incorporated by reference herein. In certain embodiments, the antimicrobial peptide has fewer than about 50 amino acids, fewer than about 25 amino acids, fewer than about 20 amino acids, fewer than about 17 amino acids, fewer than about 15 amino acids, fewer than 12 amino acids, fewer than 10 amino acids, or fewer than 9 amino acids. In certain embodiments, the antimicrobial peptide has more than about 6 amino acids, particularly more than about 7 amino acids.

In certain embodiments, the aerogel further comprises polypyrrole or magnetic nanoparticles (e.g., iron nanoparticles such as Fe₃O₄ nanoparticles). In certain embodiments, the polypyrrole or magnetic nanoparticle is conjugated (e.g., directly or via a linker (e.g., crosslinker)) to the nanofibers and/or microfibers of the aerogel. In certain embodiments, the polypyrrole or magnetic nanoparticle are packaged or contained within the aerogel (e.g., without direct conjugation to the nanofibers and/or microfibers). In certain embodiments, the polypyrrole or magnetic nanoparticles are administered with but not incorporated into the aerogel.

The aerogels of the instant invention may comprise and/or encapsulate cells or tissue (e.g., the aerogels may be seeded with cells (e.g., in the pores)). In certain embodiments, the cells are autologous to the subject to be treated with the aerogel. The aerogels may comprise and/or encapsulate any cell type. Cell types include, without limitation: embryonic stem cells, adult stem cells, bone marrow stem cells, induced pluripotent stem cells, progenitor cells (e.g., neural progenitor cells), embryonic like stem cells, mesenchymal stem cells, bone marrow mesenchymal stem cells, CAR-T cells, immune cells (including but not limited to T cells, B cells, NK cells, macrophages, neutrophils, dendritic cells and modified forms of these cells and various combinations thereof), cell based vaccines, and cell lines expressing desired therapeutic proteins and/or genes. In certain embodiments, the cells comprise stem cells. In certain embodiments, the cells comprise bone marrow stem cells (e.g., BMCSs). In certain embodiments, the cells comprise dermal fibroblasts. In certain embodiments, the aerogel comprises and/or encapsulates cell spheroids. In a particular embodiment, the aerogel comprises and/or encapsulates tissue samples (e.g., minced tissue), such as skin tissue samples or bone samples. The cells or tissue may be cultured within the aerogel (e.g., the cells or tissue may be cultured for sufficient time to allow for growth within and/or infiltration into the aerogel). For example, the cells or tissue may be cultured with the aerogel for 1, 2, 3, 4, 5, 6, 7, 8, 9, 10, 11, 12, 13, 14, 15, 16, 17, 18, 19, 20, or more days. In certain embodiments, the cells or tissue may be cultured within the aerogels in differentiation media. In certain embodiments, the cells or tissue may be seeded into the aerogels under a vacuum. In certain embodiments, the aerogel is in solution with the cells or tissue and a vacuum is applied, thereby resulting in the seeding of the cells or tissue within the aerogel.

The nanofibers and microfibers of the aerogel can be manufactured using a variety of methods. Methods for manufacturing the nanofibers and microfibers include but are not limited to: electrospinning, wet-spinning, dry-spinning, melt spinning, template synthesis, solution blow spinning, and force spinning. In certain embodiments, the nanofibers and the microfibers are synthesized by the same technique. In certain embodiments, the nanofibers and the microfibers are synthesized by different techniques. In certain embodiments, the nanofibers are made via electrospinning. In certain embodiments, the microfibers are made via wet spinning, wet electrospinning, melt spinning, and electrospinning, particularly wet-spinning or melt electrospinning. In certain embodiments, the microfibers are made via wet spinning.

Electrospinning is a versatile technique which can process different types of materials into filaments with diameters ranging from several nanometers to several microns (Do, et al. (2015) Adv. Healthcare Mater., 4:1742). This technique has been used to fabricate membranes consisting of interlaced fibers (Jiang, et al. (2015) Prog. Polym. Sci., 46:1; Kai, et al. (2014) Mater. Sci. Eng. C-Mater., 45:659; Karuppuswamy, et al., (2014) Appl. Surf. Sci., 322:162). These fibrous membranes capable of mimicking natural extracellular matrix (ECM) architectures provide biophysical and biochemical supports for cells (Caralt, et al. (2015) Am. J. Transplant 15:64; Bonnans, et al. (2014) Nat. Rev. Mol. Cell Biol., 15:786; Xu, et al. (2013) Biomaterials 34:130). Furthermore, the fibers are easily functionalized with bioactive molecules such as peptides, proteins, DNAs, and RNAs through either encapsulation or surface immobilization (Hu, et al. (2014) Control J. Release 185:12; Lec, et al. (2011) Acta Biomater., 7:3868; Xu, et al. (2008) Eur. J. Pharm. Biopharm., 70:165; Li, et al. (2010) Colloids Surf. B 75:418; Jiang, et al. (2008) Biomacromolecules 9:2097; Duque, et al. (2016) Biomaterials 106:24; Chen, et al. (2018) Adv. Drug Deliver. Rev., 132:188).

Generally, “wet spinning” refers to a process of preparing polymer fibers in which a stream of a solution comprising a polymer is ejected or extruded into a liquid bath containing a non-solvent (e.g., coagulation bath), resulting in the formation of a polymer fiber. Typically, the polymer fiber (e.g., polymer tow) is subjected to a tensile force-such as a rolling drum—that draws the polymer fiber out of the liquid bath.

Compositions comprising at least one aerogel are also encompassed by the instant invention. In certain embodiments, the composition further comprises a carrier such as a pharmaceutically acceptable carrier. In certain embodiments, the composition comprises a drug and/or bioactive agent.

In accordance with the instant invention, methods of synthesizing aerogels are provided. In certain embodiments, the method comprises freeze drying a composition comprising nanofibers and microfibers as described herein. In certain embodiments, composition is a suspension, particularly a homogenized suspension, comprising the nanofibers and microfibers. In certain embodiments, the composition is frozen within a mold (e.g., a mold of the shape desired for the synthesized aerogel (e.g., a 3D printed mold)).

After synthesis, the aerogels may be washed or rinsed in water and/or a desired carrier or buffer (e.g., a pharmaceutically or biologically acceptable carrier). The aerogels may also be stored in a cold solution, lyophilized and/or freeze-dried. The aerogels may also be physically manipulated such as by compressing and/or shaping or trimming of the aerogels (e.g., to achieve a desired shape).

In certain embodiments, the method further comprises crosslinking the aerogel. As explained herein, crosslinking may be done using a variety of techniques including thermal crosslinking, chemical crosslinking, UV-crosslinking, and photo-crosslinking. For example, the aerogel of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In certain embodiments, the aerogel is crosslinked by exposing and/or contacting the aerogel to glutaraldehyde (e.g., glutaraldehyde vapor).

In certain embodiments, the method further comprises sterilizing the aerogel. In certain embodiments, the aerogel is sterilized after crosslinking. The aerogel can be sterilized using various methods including, without limitation: gas sterilization (e.g., by treating with ethylene oxide gas), gamma irradiation, or ethanol (e.g., 70% ethanol). In certain embodiments, the method further comprises sterilizing the aerogel by exposing and/or contacting the aerogel to ethylene oxide gas.

The methods of the instant invention may further comprise synthesizing the nanofibers and microfibers of the aerogel. As explained herein, the nanofibers and microfibers of the aerogel can be manufactured using a variety of methods. Methods for manufacturing the nanofibers and microfibers include but are not limited to: electrospinning, wet-spinning, dry-spinning, melt spinning, template synthesis, solution blow spinning, and force spinning. In certain embodiments, the nanofibers and the microfibers are synthesized by the same technique. In certain embodiments, the nanofibers and the microfibers are synthesized by different techniques. In certain embodiments, the nanofibers are made via electrospinning. In certain embodiments, the microfibers are made via wet spinning, wet electrospinning, melt spinning, and electrospinning, particularly wet-spinning or melt electrospinning. In certain embodiments, the microfibers are made via wet spinning.

The methods of the instant invention may also comprise cutting or shortening the synthesized nanofibers and/or microfibers. The synthesized nanofibers and/or microfibers may be in the form of a membrane or mat. As explained herein, the nanofibers and/or microfibers of the aerogel may be short segments derived from longer fibers. The nanofiber and/or microfiber segments can be synthesized by any method. Methods of cutting or shortening the synthesized nanofibers and/or microfibers include, without limitation, cryocutting (e.g., with a cryotome), breaking, cutting, wet milling, cryomilling (e.g., cryomilling in liquid nitrogen), and homogenizing (e.g., by sonication, particularly probe sonication (e.g., ultrasonic probe sonicator, such as with a ⅛ mm probe tip)). In certain embodiments, the nanofibers and/or microfibers are cut, for example, with a knife, razor, scissor, or laser. In certain embodiments, the nanofibers and/or microfibers are frozen prior to cutting or shortening. In certain embodiments, the nanofibers and/or microfibers are frozen (e.g., in a liquid such as water) at −80° C. and then cut with a cryotome into sections (e.g., at or below −20° C.), wherein section thickness can be set to a desired amount. In certain embodiments, the nanofibers and/or microfibers, particularly the microfibers, are cut with a knife, razor, or scissor (e.g., surgical scissor). The method may further comprise filtering and/or freeze drying the cut or segmented nanofibers and/or microfibers. The method may further comprise preparing a suspension of the nanofibers and/or microfibers. The method may further comprise homogenizing (e.g., with a probe homogenizer) the suspension of the nanofibers and/or microfibers.

The methods of the instant invention may also further comprise treating the nanofibers and/or microfibers with plasma (e.g., air plasma or oxygen plasma).

The methods of the instant invention may also further comprise adding a coating material (e.g., binder) to the nanofiber and/or microfiber. In certain embodiments, the coating material (e.g., binder) is added after plasma treatment. The coating material may be applied to by any method. For example, the coating material may be applied to the aerogel by immersing or soaking the nanofiber and/or microfiber in a solution or suspension comprising the coating material. In certain embodiments, the coating material or binder is gelatin. In certain embodiments, the method comprises mixing the nanofiber and/or microfiber in a solution of the coating material (e.g., gelatin) and, optionally, homogenizing the suspension (e.g., with a probe homogenizer).

The methods of the instant invention may further comprise adding a drug and/or bioactive agent and/or cell and/or tissue to the aerogel. As explained herein, the method may comprise incorporating the drug and/or bioactive agent to and/or within the aerogel. The method may also comprise adding cells and/or tissue to and/or within the aerogel (e.g., culturing the aerogels with cells to allow for infiltration). In certain embodiments, the drug and/or bioactive agent is present prior to aerogel formation (e.g., the drug and/or bioactive agent is added to the suspension of the nanofibers and/or microfibers and/or linked (e.g., directly or via a linker) to the nanofibers and/or microfibers.

The methods of the instant invention may further comprise adding polypyrrole or magnetic nanoparticles (e.g., iron nanoparticles such as Fe₃O₄ nanoparticles) to the aerogel. As explained herein, the method may comprise incorporating the polypyrrole or magnetic nanoparticles (e.g., iron nanoparticles such as Fe₃O₄ nanoparticles) to and/or within the aerogel. In certain embodiments, the polypyrrole or magnetic nanoparticles (e.g., iron nanoparticles such as Fe₃O₄ nanoparticles) is present prior to aerogel formation (e.g., the polypyrrole or magnetic nanoparticles (e.g., iron nanoparticles such as Fe₃O₄ nanoparticles) is added to the suspension of the nanofibers and/or microfibers and/or linked (e.g., directly or via a linker) to the nanofibers and/or microfibers.

In accordance with the instant invention, methods of inhibiting, treating, and/or preventing a disease or disorder are provided. The aerogels of the instant invention may be used in inducing and/or improving/enhancing wound healing and inducing and/or improving/enhancing tissue regeneration. The aerogels of the present invention can be used for the treatment, inhibition, and/or prevention of any injury or wound. In certain embodiments, the injury or wound is a skin injury or skin lesion. In certain embodiments, the injury or wound is a caused by diabetes or is a diabetic wound. The aerogels can be used to induce, improve, or enhance wound healing associated with surgery (including non-elective (e.g., emergency) surgical procedures or elective surgical procedures). In certain embodiments, the method comprises applying or inserting an aerogel of the instant invention into a desired site (e.g., a disease site or a site of injury or bleeding). In certain embodiments, the aerogels of the present invention can be used for the inhibition, treatment, and/or prevention of a wide variety of injuries and wounds. In certain embodiments, the aerogels are used to treat hemorrhage. In certain embodiments, the aerogels are used to improve hemostasis and/or control bleeding. In certain embodiments, the aerogels are used to treat hemorrhage or bleeding in elective surgical procedures including, but not limited to: cardiac procedures, vascular procedures, transplants, oncologic procedures, liver resection, partial nephrectomy, cholecystectomy, vascular suture line reinforcement and neurosurgical procedures. In certain embodiments, the aerogels are used to treat hemorrhage or bleeding in non-elective situations including, but not limited to: splenic injury, liver fracture, large vessel laceration, cavitary wounds, epistaxis (nosebleed), minor cuts, punctures, gunshot wounds, and shrapnel wounds. In certain embodiments, the aerogels are used to treat hemorrhage or bleeding from the uterus and/or vagina (e.g., as a feminine hygiene product (e.g., tampon). In certain embodiments, the aerogels are used to absorb bodily fluids (e.g. absorption of intestinal fluid after perforative intestinal injury) (e.g., for collection of biological specimens (e.g., bacteria, virus, cells, fluids) from different locations of human bodies (e.g., gastrointestinal tract, nasal cavity, oral cavity, wound, vagina)). The aerogel of the present invention can also be incorporated into delivery devices that allow for their injection/delivery directly into a desired location (e.g., a wound). The aerogel also may be delivered directly into a cavity (such as the peritoneal cavity) (e.g., using a pressurized cannula, syringe, etc.). Due to their compressibility and flexibility, aerogels of the present invention can be incorporated into delivery devices such as syringes (automated and non-automated) that allow for their injection/delivery directly into a wound (such as a gunshot wound) or internally into a body cavity (such as the peritoneal cavity) to treat internal bleeding.

As explained herein, the aerogels can be made into any shape (e.g., by designing and utilizing a mold with the desired shape). In certain embodiments, the aerogels are manufactured into shapes such as pads, pellets, tubes, beads, etc.

The aerogels of the instant invention can be used for hemostasis, particularly junctional hemorrhage and/or non-compressible hemorrhage. In certain embodiments. the aerogels are used to manage and/or severe hemorrhage (e.g., both junctional and intra-abdominal non-compressible). In certain embodiments, the aerogels can be used to treat wounds that are incurred in the field by, for example, first responders in both military and civilian environments. In certain embodiments, the aerogels of the instant invention are used by health providers in trauma hospitals. In certain embodiments, the aerogels comprise drugs or bioactive agents which enhance healing and regenerative mechanisms, such as in acute traumatic or surgical wounds (e.g., antibiotics, analgesics, growth factors, cytokines, clotting factors, etc.).

For junctional hemorrhage, there is only one product XSTAT® (RevMedx, revmedx.com) that is currently approved for clinical use. However, XSTAT® is nonbiodegradable and needs to be completely removed at some point after implantation. The biodegradable hybrid aerogels of the instant invention have similar in vitro hemostatic efficacy as XSTAT® and have mechanical stiffness (important for tamponade of hemorrhage) that is comparable or superior to XSTAT®. Of importance, there currently is no approved product for direct treatment of non-compressible hemorrhage. The aerogels of the instant invention can be used for management of non-compressible hemorrhage as well.

The aerogels of the present invention can also be used for engineering soft tissues, tissue modeling, and/or tissue regeneration. The aerogels can be used to induce rapid tissue ingrowth, extracellular matrix deposition, and to neovascularize the tissue. In certain embodiments, the aerogels can be delivered via minimally invasive procedures for engineering soft tissues. In certain embodiments, the aerogels of the present invention can be tailored to achieve desired properties, such as magnetically responsive or electrically conductive for specific applications, including pressure sensing and actuation. Compared to aerogels made of pure nanofibers, the hybrid aerogels of the instant invention can enhance cellular infiltration. In addition, the hybrid aerogels have the potential for controlled release of small molecules (e.g., antibiotics, analgesics, growth factors, cytokines, clotting factors, etc.) which can enhance healing and regenerative mechanisms.

The aerogels of the present invention may be administered by any method. The aerogels described herein may be administered to a subject or a patient as a pharmaceutical composition. In certain embodiments, the aerogels are administered dry or lyophilized. The compositions of the instant invention comprise an aerogel and a pharmaceutically acceptable carrier. The term “patient” as used herein refers to human or animal subjects. These compositions may be employed therapeutically, under the guidance of a physician.

The compositions of the instant invention may be conveniently formulated for administration with any pharmaceutically acceptable carrier(s). For example, the areogels may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Except insofar as any conventional media or agent is incompatible with the agents to be administered, its use in the pharmaceutical preparation is contemplated.

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCI, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following examples illustrate certain embodiments of the invention. It is not intended to limit the invention in any way.

### Example 1

### Materials and Methods

### Fabrication of Hybrid Aerogels

The hybrid aerogels were fabricated using a previously unreported technique that combines electrospinning, wet electrospinning, freeze-casting, and crosslinking. The first step was the fabrication of short nanofibers (NFs) from a NF mat. In brief, 1 g of poly(ε-caprolactone) (PCL) pellets (Sigma Aldrich, St. Louis, MO) and 0.005 g of Pluronic® F-127 (Sigma Aldrich) were dissolved in 10 ml of solutions made with κ ml of dichloromethane (DCM) (99% ACS Reagent Grade, MDL Number: MFCD00000881, Oakwood Chemical, Estill, SC) and 2 ml of dimethylformamide (DMF) (ACS grade, Number: 046776, CAS: 68-12-2, Lot No: 046776P28F, MW: 73.10, Oakwood Chemical). The prepared solution was loaded into a 10 ml syringe for electrospinning. The solution was successively pumped at a flow rate of 0.7 ml/hour using a syringe pump (Fisher Scientific, Pittsburgh, PA) and electrospun under a potential of 15-18 kV between the spinneret (21 Gauge needle) using a high voltage generator (ES304-5W/DAM, Gamma High Voltage Research Inc, Ormond Beach, FL). A grounded mandrel (10 cm long and 12 cm in diameter) with the rotating speed of 1200 rpm was used to collect nanofibers. The distance between the needle and collector was 13 cm. The NF mat was removed from the collector by a razor. The NF mat was treated by air plasma and segmented by cryostat cutting. The resultant short NFs were then freeze-dried and kept at 4° C. for further use.

The second step was to prepare the short microfibers (MFs) by wet spinning. A 20% w/v PCL solution was prepared by dissolving 4 g PCL pellets in 20 ml of DCM/DMF solution (1/1 v/v). The solution was then loaded into a 20 ml syringe and extruded through a 3D printed 5-emitter extrusion device with 21-gauge needles at a rate of 3.0 ml/hour into a coagulation bath of 70% room temperature ethanol using a customized wet spinning device. The multiple emitter device was designed with five outlets and one inlet and was printed using a digital light processing 3D printer (Vida, EnvisionTEC, Gladbeck, Germany) and Clear Guide material (EnvisionTEC). A low-speed mandrel positioned above the ethanol bath was used to collect the solidified MFs. The MF bundle was allowed to dry completely after completing the wet spinning process. The dried MF bundle was cut crosswise using a surgical scissor under liquid nitrogen to avoid pressure-induced fusion of MFs. Finally, short NFs and MFs were treated with air plasma using a High Power Expanded Plasma Cleaner, specifically the PDC-001-HP model (Harrick Plasma, New York), with a voltage rating of 115 V. Subsequently, the fibers were dispersed in water to form suspensions.

To prepare short NF and MF suspensions, the dispersed fibers were then homogenized using a probe homogenizer at an amplitude of 20% with 20/10 seconds on/off cycles for 1 hour in ice-cold conditions. Next, short NF and MF suspensions were mixed in the presence of 1% gelatin (derived from bovine skin, Type A, Sigma Aldrich). This suspension was homogenized for 1 hour at the conditions mentioned above. The homogenized suspension was then poured a copper mold glued to an aluminum plate and immediately moved to a −80° C. refrigerator and kept overnight. Upon freeze-drying, the samples were crosslinked under glutaraldehyde vapor (EM grade, 2.5% in Anhydrous Ethanol, Ladd Research, Cincinnati, OH) for 24 hours, followed by ethylene oxide gas (Anprolene AN7916 Ethylene Oxide Ampoules, Andersen Sterilizers Inc., Haw River, NC) sterilization.

### Characterization

The dimensions (length and diameters) of short NFs and MFs were calculated using Image J software (NIH). The cross-sectional morphology of hybrid aerogels was observed by scanning electron microscopy (SEM, FEI Quanta 200, Hillsboro, OR) at a standard operating condition (accelerating voltage: 25.0 kV, spot: 3.0, and dwell time: 1-3 μs).

The mass and dimensions of cylindrical aerogels were measured using a digital balance and caliper with a resolution of 0.0001 g and 0.01 mm, respectively. The density of aerogels was calculated by the following equation (Liu, et al. (2019) ACS Nano 13:2927-2935):

$$ρ ( g . {cm}^{- 3} ) = m / V$$

where ρ is the density and m and V represent aerogels' mass and volume. The volume of the aerogels was calculated by the following equation:

$$V = {\int }_{a}^{b} A ( x ) dx or {\int }_{a}^{b} ( A 1 ( x ) - A 2 ( x ) ) dx )$$

The volume of the cylindrical aerogels was calculated by the equation: V=πr²h, where r and h represent the radius and height of aerogels, respectively. In addition, the porosity (ε) of 3D aerogels was estimated based on the following equation.

$$ε = ( V - {V}_{0} ) / V \times 100 \%$$

where V₀=m₀/ρ₀ is the calculated volume of bulk materials, m₀ is the mass of bulk materials, and ρ₀ is the density of bulk materials. Additionally, the porosity and pore size of the test samples were assessed using a microCT analysis (Bruker SkyScan 1276-CMOS Edition, Kartuizersweg, Kontich, Belgium).

Next, a structure tensor tool was used to analyze the orientation of NFs and MFs within the hybrid aerogels. The resulting aerogels were frozen in ice and cut using a razor to avoid the fiber deformation during cutting. Each sample was mounted on an SEM imaging stub with silver paste. SEM images were taken at different magnifications for each sample and individual fiber alignment was measured. Distribution of fiber orientation was determined with a structure tensor using a 2-pixel Gaussian window and Gaussian Gradient. The structure tensor at location x₀ in the cross-sectional view of hybrid aerogels is defined by the following equation (Jähne, B. Digital Image Processing: Concepts, Algorithms, and Scientific Applications (Springer Berlin Heidelberg, 1995, doi: 10.1007/978-3-662-03174-2):

$$J ( {x}_{0} ) = {\int }_{R 2}^{1} w ( x - {x}_{0} ) ( ∇ f ( x ) ) {∇}^{T} f ( x ) d {x}_{1} d {x}_{2}$$

where w is a nonnegative isotropic observation window (e.g., a Gaussian) centered at x₀. Two measures were defined to estimate the orientation of the fibers by the J: coherence (C) and energy (E). The coherence, C, was computed by the following equation:

$$0 \leq C = ( {λ}_{max} - {λ}_{min} ) / ( {λ}_{max} + {λ}_{min} ) = √ ( {( {J}_{22} - {J}_{11} )}^{2} + 4 {J}_{12}^{2} ) / ( {J}_{22} - {J}_{11} ) \leq 1$$

where the eigenvalues of the structure tensor are noted as λₘₐₓ and λₘᵢₙ. Furthermore, the energy of the fibers in the direction θ was computed by the following equation:

$${‖ {D}_{θ} f ‖}_{w}^{2} = {( {θ}^{T} ∇ f , {θ}^{T} ∇ f )}_{w} = ( {{θ}^{T} ( ∇ f , ∇ f )}_{w} θ = {θ}^{T} {J}_{θ}$$

where ∥D_{θ}f∥_{w}² is the average energy in the window (w).

### Fluid-Responsive Shape-Memory Properties

The shape-memory properties of different-shaped aerogels were measured according to a previously published protocol (Du, et al. (2021) Nat. Commun., 12:4733). NFA, MFA, NF/MF-A1, NF/MF-A2, or NF/MF-A3 aerogels were compressed to achieve the shape-fixed state. Next, the aerogels were put into contact with water or solutions with different pHs in a shape-fixed state, and their shape-memory properties were recorded by a digital camera (Samsung M30S). The aerogels' initial dimensions for testing shape-memory in water were 10 mmin diameter and 30 mm in height, whereas, for shape-memory testing in different pH solutions, the dimensions were 10 mm in diameter and 10 mm in height. At different time points, their shape was measured by a digital caliper. In addition to the compression resistance test, their maximum bending resistance was also measured by bending the top part of the aerogels. A digital camera recorded the resistance against external pressure. The external compression release properties of soaked aerogels were also measured by compressing the aerogels on the top. The amount of released water upon compression and the reabsorbed water upon releasing the force were weighed by a digital balance and recorded by a digital camera (Samsung M30S). The heart-shaped aerogels went through 72 cycles of compression and relaxation per min, like healthy human heart which beats 72 times per minute. The breast-shaped aerogels were tested for 100 compression-relaxation cycles to demonstrate their flexibility. The shape-memory properties of the human heart and breast-shaped aerogels were recorded by a digital camera (Samsung M30S).

### Mechanical Properties

The aerogels were first examined by a compression test. Aerogels in a cylindrical shape (diameter: 3 mm and height: 10 mm) were tested using an Instron 5640 universal test machine. After fixing the dry samples on the lower compression plate of a CellScale Univert with double-sided tape, a 200 N load plate-initiated compression to reach 50%, 70%, and 90% displacement. To calculate the shape recovery of the fixed samples, all aerogels were soaked in water after 50%, 70%, and 90% compression and their length before and after recovery were measured using a digital caliper. Young's modulus was calculated using the following equation (Zhang, et al. (2022) Biomaterials 285:121546; Wang, et al. (2022) Adv. Mater., 34:2108325):

$$Young ’ s Modulus = \{ ( F / A ) / ( Δ H / {H}_{0} ) \} = {FH}_{0} / A Δ H$$

where F, A, H₀, and ΔH represent the compressive force, cross-sectional area, initial height, and change in the height of aerogels, respectively.

For the cyclic compression test, all experiments were carried out in a water bath to prevent water from evaporating from the aerogels (Sun, et al. (2013) Nat. Mater., 12:932-937). Samples underwent the cyclic compression tests for 10 cycles at 50%, 70%, and 90% strain for each cycle. NF/MF-A1 were tested for 100 cycles to demonstrate their mechanical robustness. The durations for compression, hold, recovery, and rest were set for 10, 5, 10, and 5 seconds for each cycle. It is worth noting that the force was not kept constant during the cyclic compression and relaxation cycles because the force was measured at different strains. Instead, the rate of compression or relaxation was set to 1 mm/s, and force measurements were taken at various strains (i.e., 50%, 70%, and 90%) during compression. However, NF/MF-A1 were tested for 100 cycles to demonstrate their mechanical robustness at a rate of 2 mm/s. The effect of compressive strains on the mechanical strength of aerogels was tested. The experiment involved subjecting a sample to cyclic compressive strains of 50%, 70%, and 90% for a total of five repetitive rounds. Each round consisted of three repetitive cycles under three different compressive strains, with each cycle including 15 seconds of compression, 5 seconds of hold, 15 seconds of recovery, and 5 seconds of rest. The sample was initially subjected to the 50% compressive strain, followed by 70% and 90% compressive strains to complete the first round. Subsequently, the same sample was subjected to four more rounds of cyclic compression test under the same condition.

Next, critical forces, max forces, and force loss were computed to determine comparable metrics of compression resistance between aerogels. First, critical force is defined as the force required to initiate compression. Critical force was computed as the maximum force reached the top of the force-displacement sliding curve. Similarly, max force is defined as the maximum compressive force achieved during a 90% displacement. Finally, force loss is defined as the percentage difference between max forces in successive cyclic compressions. The change in length for different aerogels after cyclic compression tests is defined as the percentage difference between max changes in height in successive cyclic compressions.

To examine the fracture energy of hybrid aerogels in atmosphere, all tested samples were cut into 50 mm long, 20 mm width, and 4 mm thickness. The two arms of a test sample were clamped, and then the upper arm was pulled upward at a deformation rate of 2 mm/s while the tearing force was recorded. The fracture energy was calculated by the following equation (Sun, et al. (2013) Nat. Mater., 12:932-937):

$$E = 2 F / W$$

where E is the fracture energy, F is the maximum force from the test, and W is the thickness of the samples (Sun, et al. (2013) Nat. Mater., 12:932-937). The stability of aerogels in wet conditions were also measured under a strong mechanical agitation (1500 rpm) for 1 hour.

### Weighted Aerogel Ranking

To evaluate the performance of the tested aerogels, a Z-score was computed, and a standardized score was used to transform raw data to make it easier to understand and normalize the range of possible values (McCarthy, et al. (2023) Adv. Mater., 35:2207335). The Z-score scale has a mean of 0 and a standard deviation of 1, which allows for comparison across different datasets. Each aerogel's performance was quantified and standardized using the Z-score formula, which subtracts the sample mean (u) from the observed value (x) and divides it by the sample standard deviation (o) as shown in the following formula (McCarthy, et al. (2023) Adv. Mater., 35:2207335):

$$Z = ( x - μ ) / σ$$

To further enhance the analysis, categorical and subjective weighting was applied to several criteria, such as compressive strength, tensile modulus, fracture energy, tearing strength, density, and toughness. By weighing each aerogel's parameter based on its relative importance, the overall performance of each aerogel was evaluated more accurately. The use of Z-scores and standardized weighting allowed for a more objective evaluation of each aerogel's performance. The weights assigned to each criterion were determined based on domain expertise and an intuitive understanding of their relative importance. By assigning standardized weights to the standardized Z-scores, the individual aerogel was ranked based on its performance, allowing for easy identification of the most effective design. This approach allowed for direct comparison among different tested aerogels and provided a clear roadmap for optimizing aerogel design for further study. The weighted average was calculated by the following equation (Price, G. R. (1972) Ann. Hum. Genet., 35:485-490):

$$x = {Σ}_{i = 1}^{n} {w}_{i} \cdot {x}_{i} / {Σ}_{i = 1}^{n} {w}_{i}$$

where x=weighted average, wᵢ=sum of the product of the weight, and xᵢ=data number.

### Minimally Invasive Delivery Properties

The minimally invasive delivery of cell-seeded hybrid aerogels and decellularized hybrid aerogels was studied. First, various hybrid aerogels (diameter×height: 8×2, 10×2, 12×2, or 7×10 mm²) were first sterilized by the ethylene oxide gas for 12 hours. Next, GFP-labeled dermal fibroblasts were seeded on aerogels. After 14 days of culture, the cell-containing aerogel was loaded into an applicator with a 1.7 mm inner diameter cannula for injection. The decellularized aerogels were dried and rolled to test their ability to be delivered in a minimally invasive manner. The injectability of GFP-labeled dermal fibroblast-containing and dry decellularized hybrid aerogels were recorded by a digital camera (Samsung M30S). The viability of the cells in hybrid aerogels before and after injection was also measured using the 3-(4,5-dimethylthiazol-2-yl)-2,5-diphenyl-2H-tetrazolium bromide (MTT) assay (Abcam, Waltham, MA).

### Cell Culture Studies

Samples were sterilized by ethylene oxide gas for 12 hours prior to in vitro cell culture studies. The human immortalized keratinocyte cell line, HaCaT, utilized in assessing cellular toxicity of the tested materials sourced from Antibody Research Corporation (Catalog No. 116027, St. Peters, MO). These cells were stored in a liquid nitrogen tank. Initially, the cells were cultured in Dulbecco's modified Eagle's medium (DMEM) (Gibco Inc., Waltham, MA) containing 10% fetal bovine serum (Abcam, Waltham, MA) and 1% antibiotics (10,000 μg/ml streptomycin and 10,000 units/ml penicillin) (ThermoFisher Scientific, Waltham, MA) at 37° C. with 5% CO₂. Next, NFA, MFA, or NF/MF-A1 (diameter=8 mm, height=2 mm) were immersed in the complete cell culture media, and 6×10⁴ HaCaT cells were seeded on each aerogel. After 36 hours of incubation, cells were stained with ethidiumhomodimer-1 (Catalog No. L3224, ThermoFisher Scientific) for dead cells in red fluorescence and Calcein-AM (Catalog No. L3224, ThermoFisher Scientific) for live cells in green fluorescence. A CLSM (Zeiss 710, Zeiss, Dublin, CA) was used to obtain the images. To construct the video, z-stake images were merged using Zeiss' Zen Blue software (Zeiss).

GFP-labeled dermal fibroblasts were also seeded on different aerogels and imaged using CLSM. Briefly, 1×10⁵ GFP-labeled dermal fibroblasts were seeded on the NFA, MFA, or NF/MF-A1 and cultured for 3 and 7 days. After culture, the cells on the aerogels were imaged. The 3D view and orthogonal images were directly constructed using Zeiss' Zen Black software (Zeiss, Dublin, CA) and used without any further modification.

To study the neurite growth, the Human Ren VM NPC cell line (Catalog No. SCC008, Millipore, Burlington, MA) and was cultured in neurobasal media supplemented with 1% MEM non-essential amino acids, 1% N2 supplement, 2% B27 supplement, 1% Glutamax, 0.01% β-mercaptoethanol and 1% penicillin-streptomycin (ThermoFisher Scientific) on Cultrex (R&D systems)-coated tissue culture plates. 1.0×10⁵ cells were seeded on NFA, MFA, and NF/MF-A1 for 7 days. On day 7, the aerogels containing the cells were washed with DPBS and fixed in 4% formalin for 5 minutes. The aerogels were washed twice and blocked with 1% BSA containing 0.1% triton-x 100 in DPBS for 30 minutes. Primary antibody (Rabbit β-III tubulin from Abcam, 1:1000 dilution, Catalog No. #ab 18207, Abcam) was added and incubated at 4° C. overnight. On the next day, the samples were washed thrice with DPBS and incubated with a secondary antibody (Goat anti-rabbit tagged with Alexa fluor 488, 1:8000 dilution, Catalog No. #ab 150077, Abcam) for 1 hour. The samples were then washed thrice and stained with Hoechst 33342 to visualize the nucleus. Z-stack images were converted to 3D view and depth coding images using Zeiss' Zen Blue software (Zeiss 710, Zeiss, Dublin, CA). Depth mapping values were automatically generated by the software and were presented unchanged. The migration of neural stem cells and axonal growth in the aerogels was measured based on 3D view and depth coding images.

To study the migration of cells throughout aerogels, GFP-labeled dermal fibroblasts were first cultured on the culture plate and formed a cell monolayer. NF/MF-A1 was placed on top of the cell monolayer. The migration of cells from the bottom surface to the top surface of aerogels was observed and visualized at different time points (0, 3, 6, and 9 days) using a CLSM (Zeiss LSM800, Zeiss, Dublin, CA) (Chen, et al. (2020) Adv. Mater., 32:2003754). The cellular migration in the scaffolds was calculated using CLSM images to determine the cell coverage area at different time points (John, et al. (2023) Adv. Funct. Mater., 33:2206936). Image J software was used to calculate cell coverage area by measuring the bottom part of the scaffolds (Ab) and the area of cell migration on the top of the aerogel (ACM), which was determined based on the fluorescence of the migrated cells. The percentage of cell coverage area was calculated by the following equation.

$$Cell Coverage Area ( \% ) = ACm / Ab \times 100$$

### Animal Studies

The animal study was conducted according to the approved guideline of the Institutional Animal Care and Use Committee (IACUC) at the University of Nebraska Medical Center (UNMC) under protocol No. 17-103-11-FC. In brief, ten weeks old male Sprague Dawley (SD) rats were purchased from Charles River Laboratories (Wilmington, MA), and were housed in the animal facility at UNMC. Rats had free access to regular laboratory food and water and were regularly monitored by staffs in UNMC Comparative Medicine. After one week of acclimation facilities, all rats (total=24) were randomly divided into two cohorts (N=12) according to the study periods (14 days and 28 days). 1-5% isoflurane through a nose cap was used for anesthesia. Rats were placed on a heating pad to maintain body temperature during the procedure. An area of 8×4 cm² on the back of each rat was shaved, and the exposed skin was treated with a povidone-iodine solution to create an aseptic environment at the surgical site. Each rat received four different types of implants: 2D NF mat, NFA, MFA, and NF/MF-A1. The dimensions of samples were 10 mm in diameter and 2 mm in height. Incisions of 1.5 cm long were made in 4 paraspinal sites on the dorsal regions of the SD rats, and one subcutaneous pocket was made on each incision using blunt dissection. Then, the implant was gently inserted through the puncture site into the subcutaneous pocket. After placement of the implants, the skin incisions were closed with staples. The rats were then removed from anesthesia to recover on the heating pad before being transferred to their cage. The rats were euthanized using CO₂ inhalation after 14 and 28 days of implantation. The dorsal skin was then carefully resected and immediately immersed in PBS solution. After that, the skin sections containing scaffolds were photographed, and the explants were processed for histological analysis.

### Histology

The isolated explants were submerged in formalin, dehydrated in a graded ethanol series of 70-100%, embedded in paraffin, and finally sectioned at a thickness of 5 μm. Hematoxylin and eosin (H&E) and Masson's trichrome (MT) staining were performed by the Tissue Science facility at UNMC. The percentage of cell infiltration area within the explants was analyzed by Image J software. New blood vessel formation was measured by the number of blood vessels per mm² of explants. The formation of new blood vessels was identified by red blood cells and endothelial lining in the explants. The granulation tissue formation was measured by quantifying the collagen deposition in the explants based on MT staining results. In brief, original MT staining images of explants were converted into RGB images, and these images were deconvoluted by image J software using the color deconvolution plugin. The green channel was identified as collagen fibers. The integrated density of green channels was then quantified (Chen, et al. (2017) Int. J. Clin. Exp. Med., 10:14904-14910).

### SEM Imaging of Dry Tissue Samples

The cross-sectional morphology of each explant type was characterized by SEM. The chemical-dry method was used to prepare the explant sample for SEM. In brief, the samples were rinsed with 1×DPBS and fixed with a tissue fixative solution made with 2% paraformaldehyde (PolySciences, Warrington, PA) and 2.5% glutaraldehyde (Ladd Research) in 0.1 M Sorenson phosphate (Electron Microscope Sciences, Hatfield, PA) or sedum cacodylate buffer (Electron Microscope Sciences) for 3 hours at room temperature. After fixation, the samples were washed with the same buffer (2×) for 5 minutes each. Then, the samples were incubated with 1% osmium tetroxide for 30 minutes at room temperature, followed by three washes with the same buffer (5 minutes each). The samples were dehydrated in a graded ethanol series (35, 50, 70, 95, and 100%) for 5 minutes each. The samples were treated in a graded hexamethyldisilazane (HMDS, Electron Microscope Sciences) series (30, 70, and 100%) for 5 minutes each. Finally, the samples were left in 100% HMDS to dry in air in the chemical hood.

Functionalization of Hybrid Aerogels with Polypyrrole and Fe₃O₄

To demonstrate the hybrid aerogels' versatility for functionalization, the polypyrrole coating method was used (Jiang, et al. (2019) Nat. Commun., 10:3491). Briefly, the aerogel (diameter× height: 10 mm×20 mm) was immersed in a 0.04M pyrrole (Sigma-Aldrich, St. Louis, MO) solution, mixed with an equal volume of a 0.084M FeCl3 (Sigma-Aldrich) solution at room temperature, and subjected to 30 minutes of ultrasonic treatment for uniform polymerization. Subsequently, a 3 V battery-powered circuit was used to measure the initial (R₀) and retained (R) resistance of the polypyrrole-coated hybrid aerogel (NF/MF/PPy) under compressive strains of 0%, 25%, 50%, 70%, and 90%. The resistance change was quantified using the equation:

$$Δ R / {R}_{0} = ( {R}_{0} - R ) / {R}_{0}$$

Additionally, Fe₃O₄ magnetic particles (Millipore Sigma, Burlington, MA) was introduced, facilitating the integration of magnet-responsive building blocks. FIG. 5K displays the schematic illustrating the absorption of Fe₃O₄ magnetic particles into the hybrid aerogels. Following water removal, the hybrid aerogels maintained their compressed shape, with the subsequent introduction of the Fe₃O₄ suspension facilitating recovery from deformation. This process led to the development of the NF/MF/Fe₃O₄ composite hybrid aerogels, and after freeze-drying the composite hybrid hydrogel enabled magnetic attraction through the embedded Fe₃O₄ particles.

### Statistical Analysis

Data were shown as mean±standard deviation. Statistical analyses were performed using analysis of variance (ANOVA) in GraphPad Prism (version 9.5.1). Pairwise comparisons were made using ordinary one-way ANOVAs with Tukey's multiple comparisons post hoc test. For cases with a large number of pairwise comparisons, a separate table was created to display each p-value, which was added to the supplemental information. To obtain measurements from photographs/SEM images, ImageJ was used after calibrating pixels to mm/μm. Statistical significance was expressed as *p<0.05, **p<0.01, **p<0.001, ****p<0.0001, and a lack of pairwise comparison bars implies a lack of significance (p>0.05, ns).

### Results

Hybrid aerogels were fabricated using a combination of electrospinning, wet spinning, fiber cutting, freeze-casting, and crosslinking. In a particular embodiment, the method comprises preparation of short NF by producing NF mats by electrospinning, cryo-cutting 2D NF mats into short NFs, treating plasma, and dispersing NFs. The method also comprises fabrication of short MF by producing MFs by wet spinning, cutting them into short MFs, treating plasma, and dispersing MFs. The method then comprises fabrication of hybrid aerogels by freeze-drying of the NF and MF suspension containing gelatin as a crosslinker/binder in a 3D-printed mold followed by crosslinking with GA vapor and removal of the mold.

In more detail, the synthesis method comprised the following. Firstly, NF mats were produced by electrospinning a solution of poly(ε-caprolactone) (PCL) in dichloromethane (DCM)/dimethylformamide (DMF) (John, et al. (2020) Small 16:1907393; Su, et al. (2020) ACS Nano 14:11775-11786). PCL was chosen as raw materials because it has been widely used in many biomedical applications (Malikmammadov, et al. (2018) J. Biomater. Sci. Polym. Ed., 29:863-893). The mats were then treated with air plasma and were cut into short NFs using cryocutting. Next, the short NFs were dispersed in water to create a short NF suspension via homogenization.

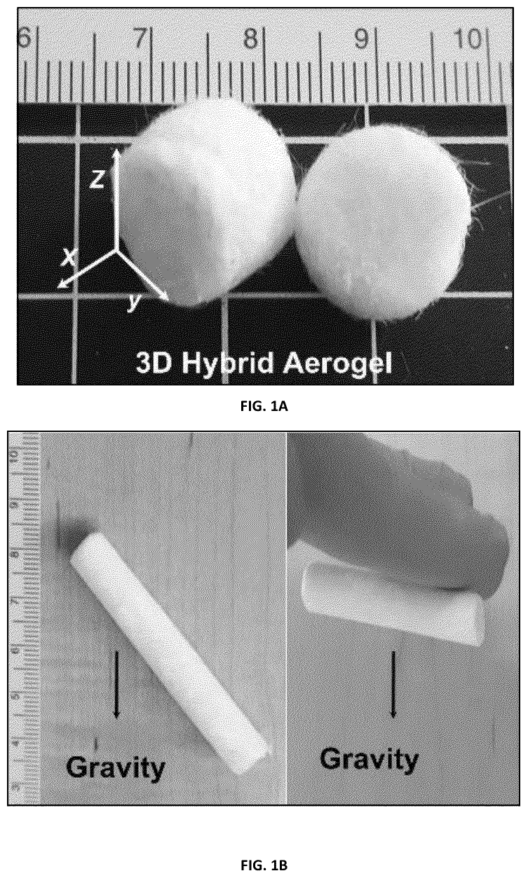

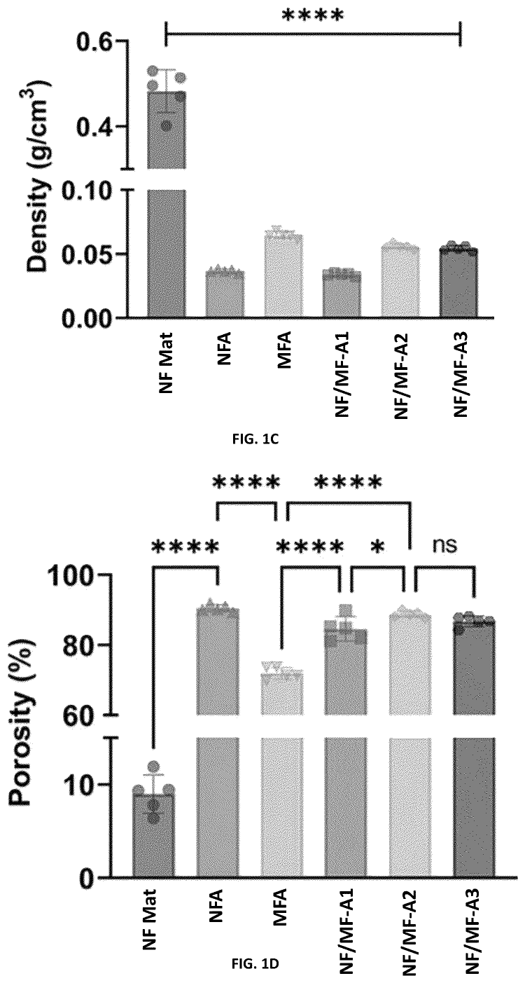

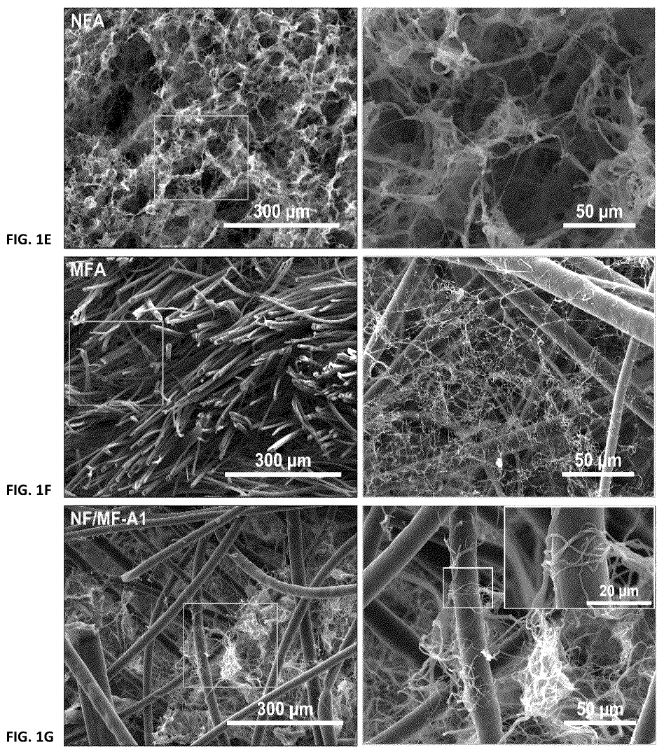

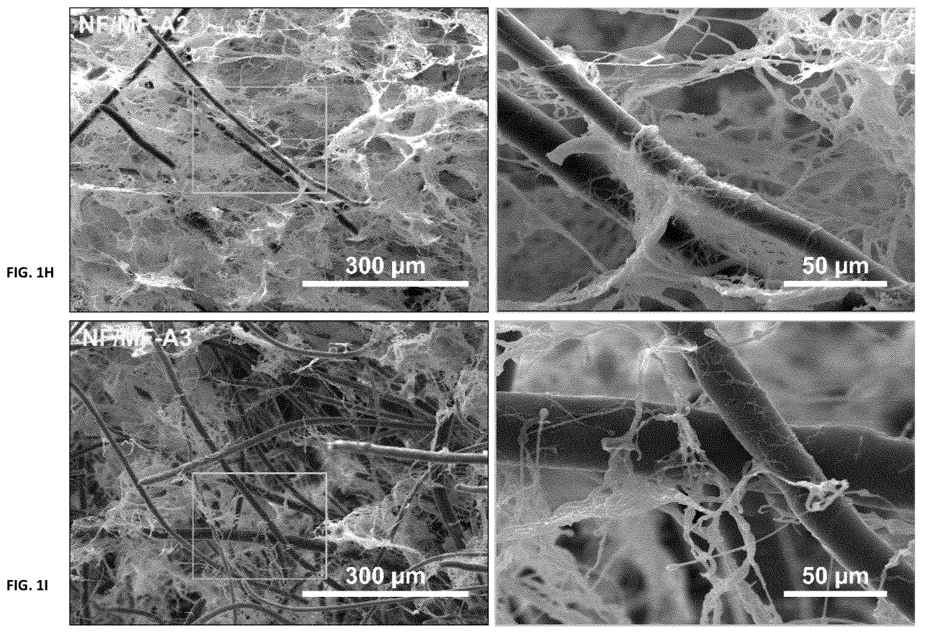

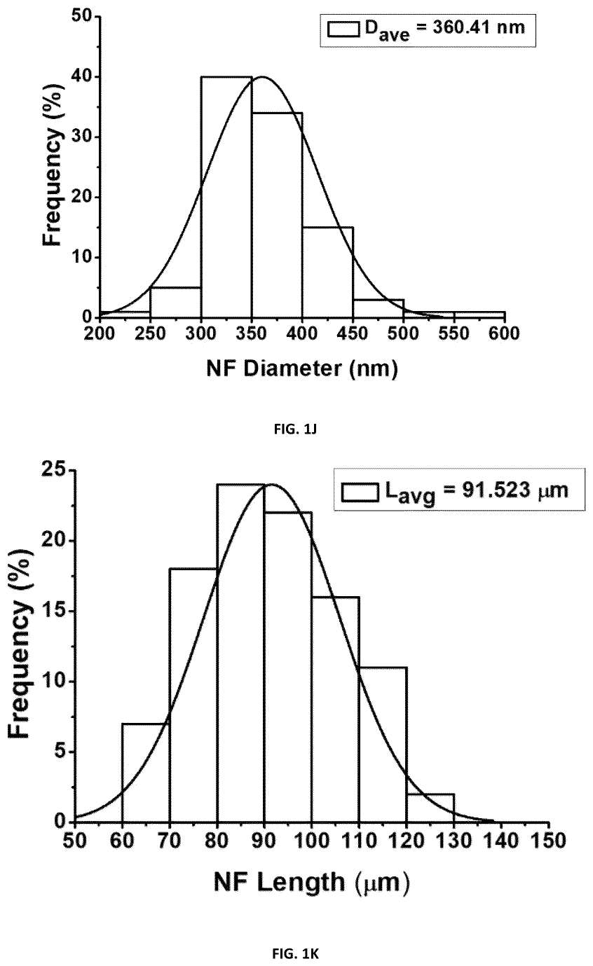

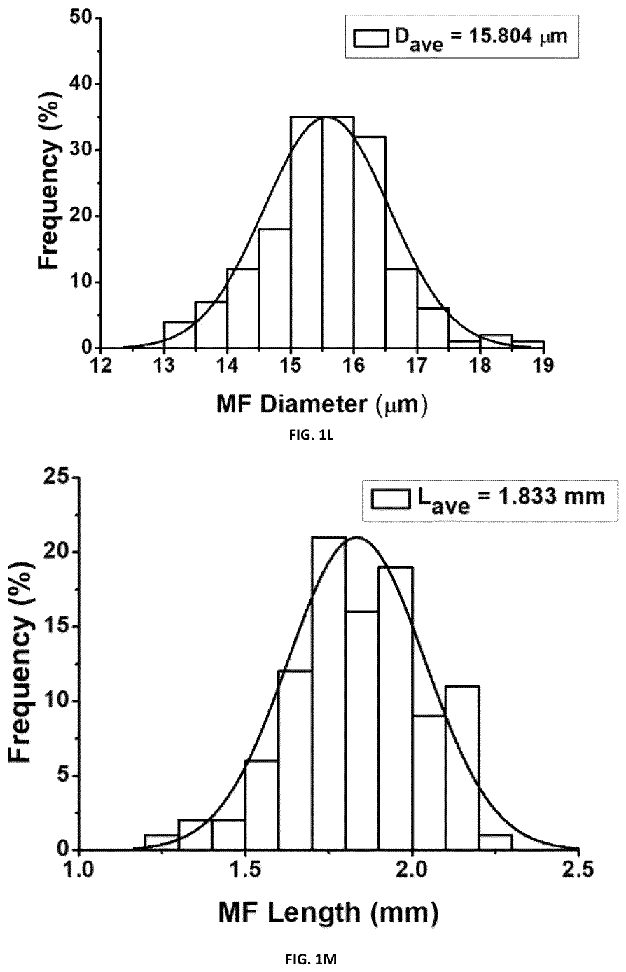

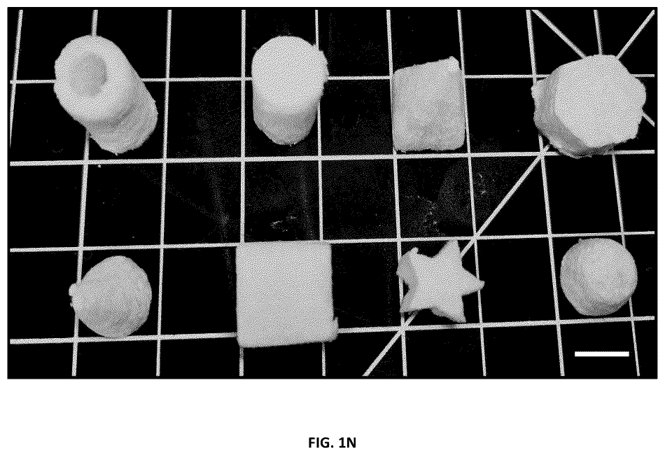

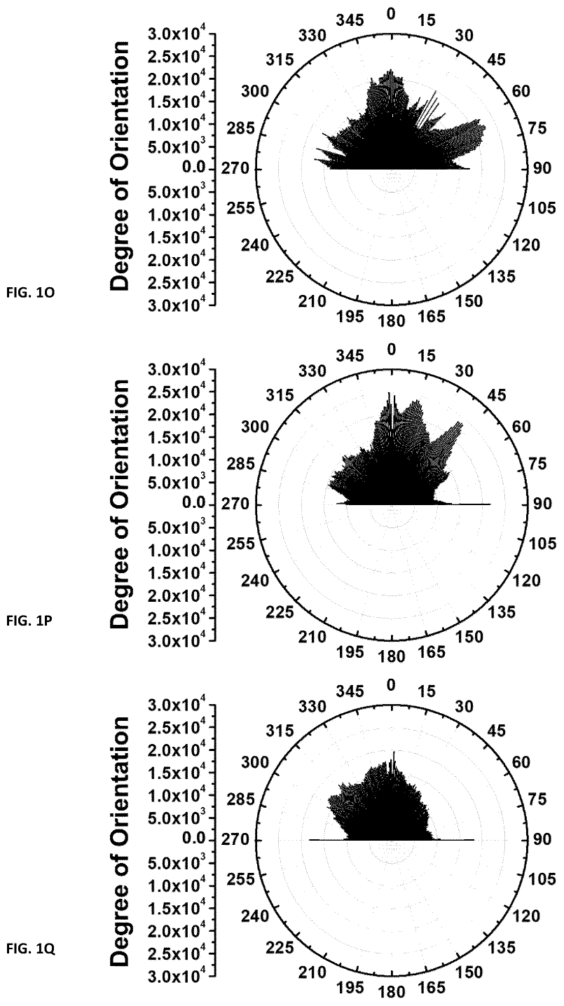

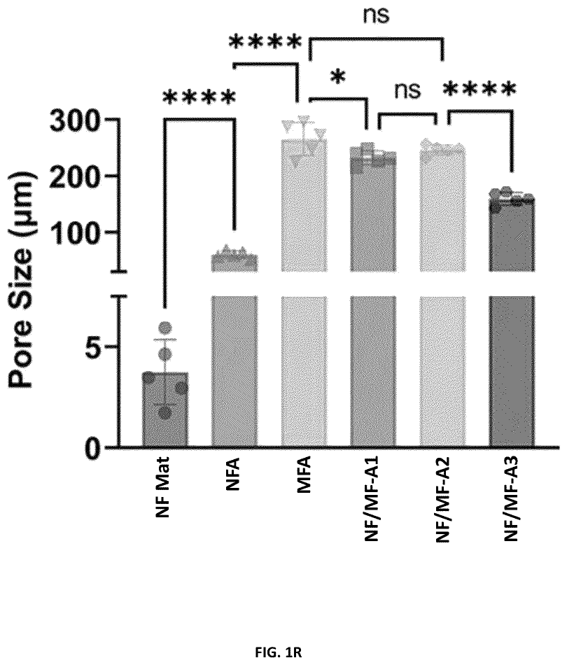

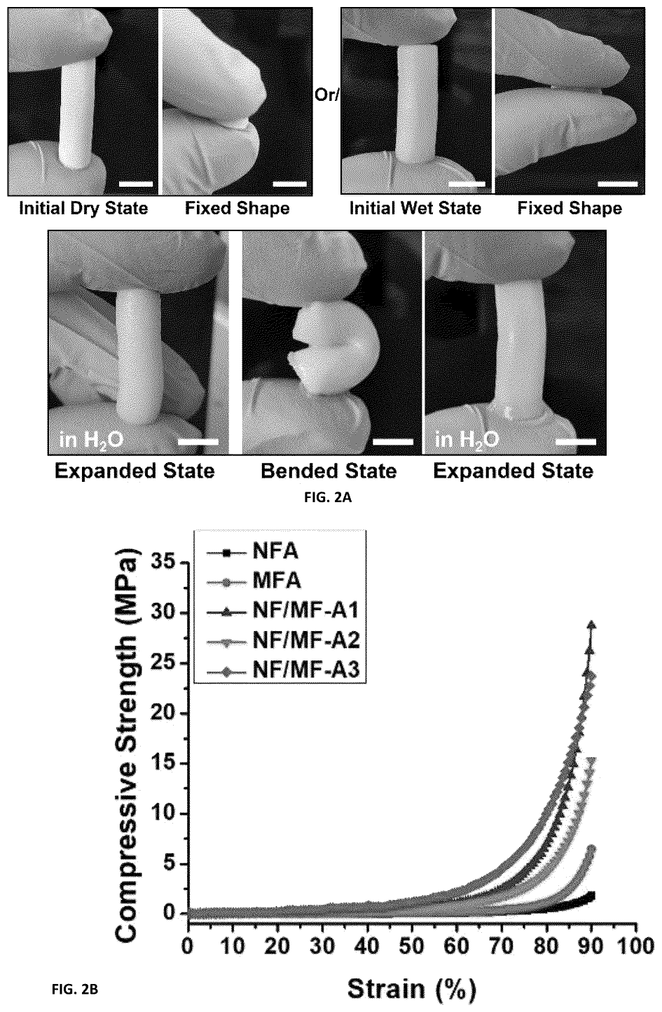

Similarly, short MF solutions were fabricated through wet spinning, plasma treatment, cutting, and dispersing (McCarthy, et al. (2021) Adv. Healthc. Mater., 10:2100766; McCarthy, et al. (2021) Mater. Today Bio., 12:100166). The average diameter and length for obtained short NFs were 360.4±54.5 nm and 91.5±14.3 μm, respectively (FIGS. 1J, 1K). The resulting short MFs had an average diameter and length of 15.8±1.1 μm and 1.9±0.2 mm, respectively (FIGS. 1L, 1M).

The short NFs and short MFs suspended in a 1% gelatin solution were homogenized in different weight ratios (e.g., 0:100, 25:75, 50:50, 75:25, 100:0), frozen at −80° C. in customized molds and freeze-dried to obtain hybrid aerogels. Finally, the hybrid aerogels were crosslinked with 2.5% glutaraldehyde (GA) vapor to stabilize the structure.

The hybrid aerogels were produced using a freeze-casting process with customized molds, resulting in various shapes such as cylinder, hollow cylinder, cone, cube (FIGS. 1A and 1N) (John, et al. (2021) Adv. Healthc. Mater., 10:2100238). The ultra-light properties of the aerogels were demonstrated by their ability to be held against gravity by electrostatic force generated between the aerogels (a volume of ˜447.7 cm³) and a wooden wall or powder-free hand gloves (FIG. 1B). The densities of the hybrid aerogels were significantly lower than those of NF mats and displayed a highly porous structure with porosity of >84%, compared to 8.9±1.8% for NF mats (FIGS. 1C and 1D). Furthermore, the 2D NF mat exhibited an average pore size of 3.7±1.4 μm, whereas the NF aerogel coated with 1% gelatin (NFA) demonstrated a larger size at 60.3+6.1 μm (FIG. 1R). Microfiber aerogel coated with 1% gelatin (MFA) displayed the largest average pore size, measuring 265.5±26.3 μm, followed closely by NF/MF-A2 (hybrid aerogels containing NF/MF at a ratio of 75:25 w/w coated with 1% gelatin), NF/MF-A1 (hybrid aerogels containing NF/MF at a ratio of 50:50 w/w coated with 1% gelatin), and NF/MF-A3 (hybrid aerogels containing NF/MF at a ratio of 25:75 (w/w) coated with 1% gelatin), with respective sizes of 246.5±8.1 μm, 232.1±11.1 μm, and 159.3±10.1 μm (FIG. 1R).

The highly porous structure is ideal for nutrition supply and waste exchange during 3D cell culture, wound healing, and tissue regeneration and fast host cell penetration (Chen, et al. (2020) Advanced Materials 32:2003754; Chen, et al. (2021) Sci. Adv. 7: cabg3089; Tran, et al. (2022) Adv. Wound Care 12:399-427). The scanning electron microscopy (SEM) images revealed the NF aerogels (NFAs) had a nest-like structures with velvety PCL NFs (FIG. 1E), while microfiber aerogels (MFAs) consisted mainly of PCL MFs with a small amount of gelatin nanofibrils (FIG. 1F). Gelatin nanofibrils could be formed due to phase separation, which was similar to the formation of GelMA nanofibrils (Chen, et al. (2021) ACS Biomater. Sci. Eng., 7:2204-2211). Interestingly, hybrid aerogels with a NF/MF weight ratio of 50:50 (NF/MF-A1) exhibit a bi-continuous fibrillar network where the soft NFs and hard MFs were physically entangled (FIG. 1G). The large pores resulting from the MF component allow cells to migrate effectively into and within aerogels, while the NF component mimics the morphology of ECM, making it suitable for 3D cell culture, wound healing, and tissue regeneration applications (McCarthy, et al. (2021) Mater. Today Bio., 12:100166). Similar morphology and entanglement was observed between NFs and MFs in hybrid aerogels with NF/MF weight ratios of 75:25 (NF/MF-A2) and 25:75 ((NF/MF-A3) (FIGS. 1H and 1I). This entanglement between NFs and MFs contributes to the superior tear resistance of the hybrid aerogels. Furthermore, the tensor analysis shows that the hybrid aerogels were composed of random fibers, contributing to their isotropic structure, which is beneficial for tear resistance (FIGS. 1O-1Q).

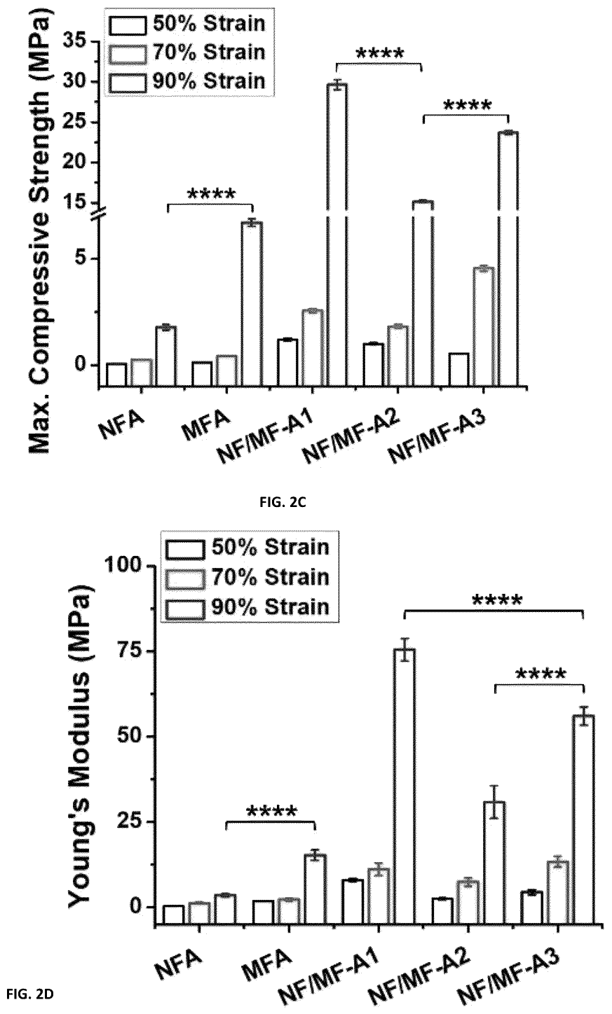

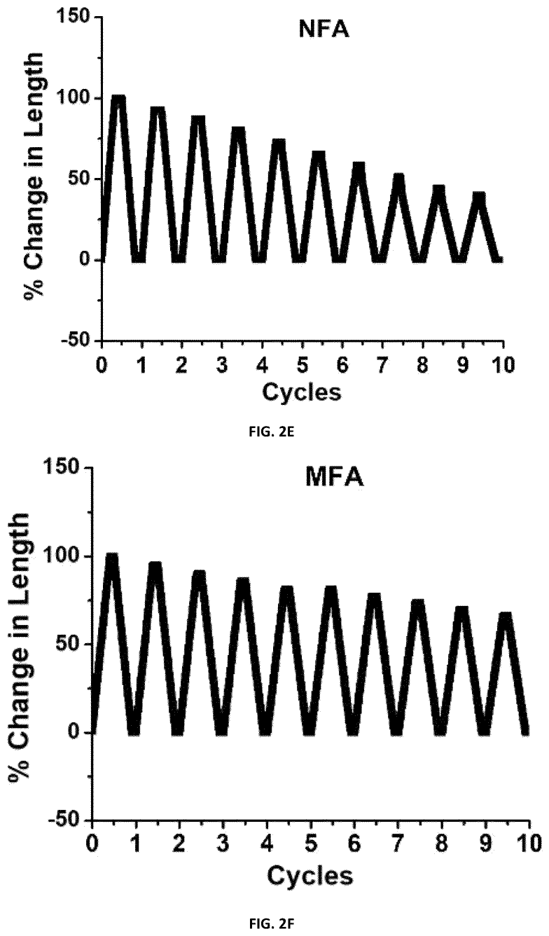

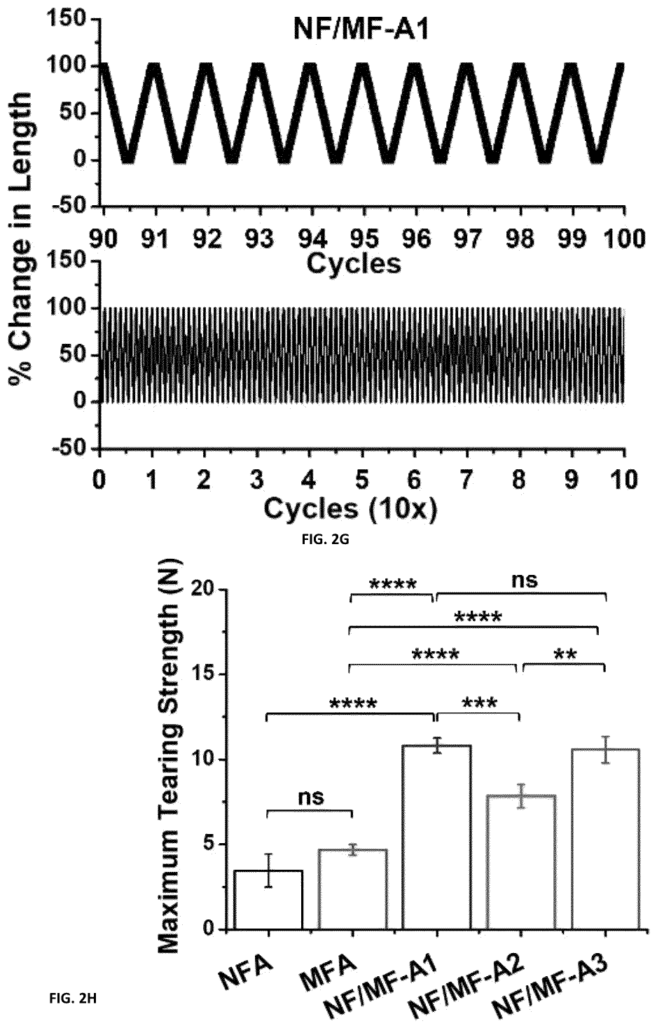

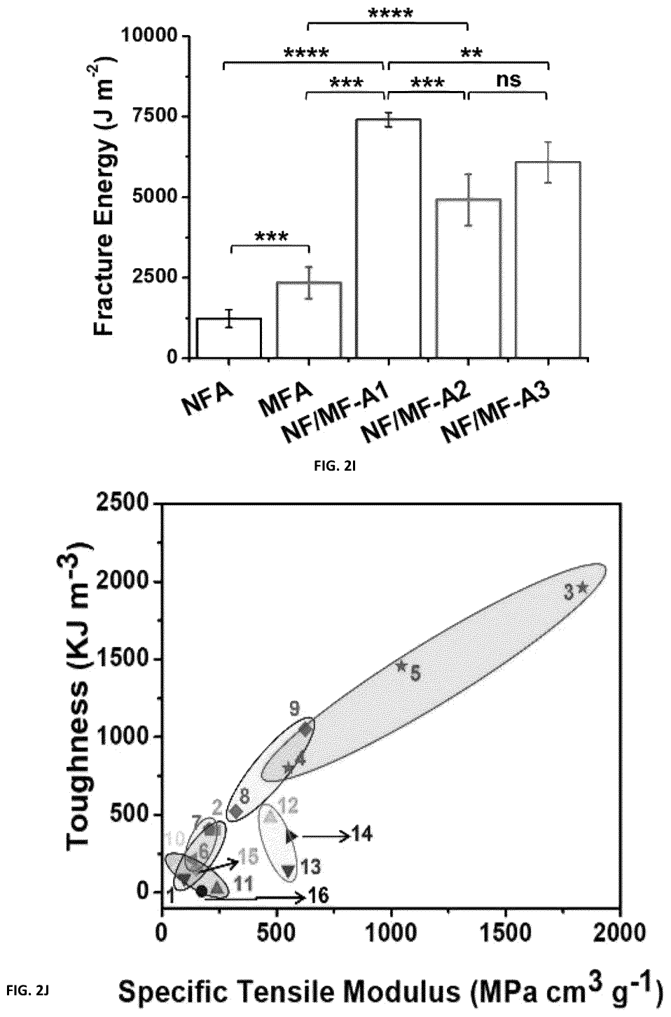

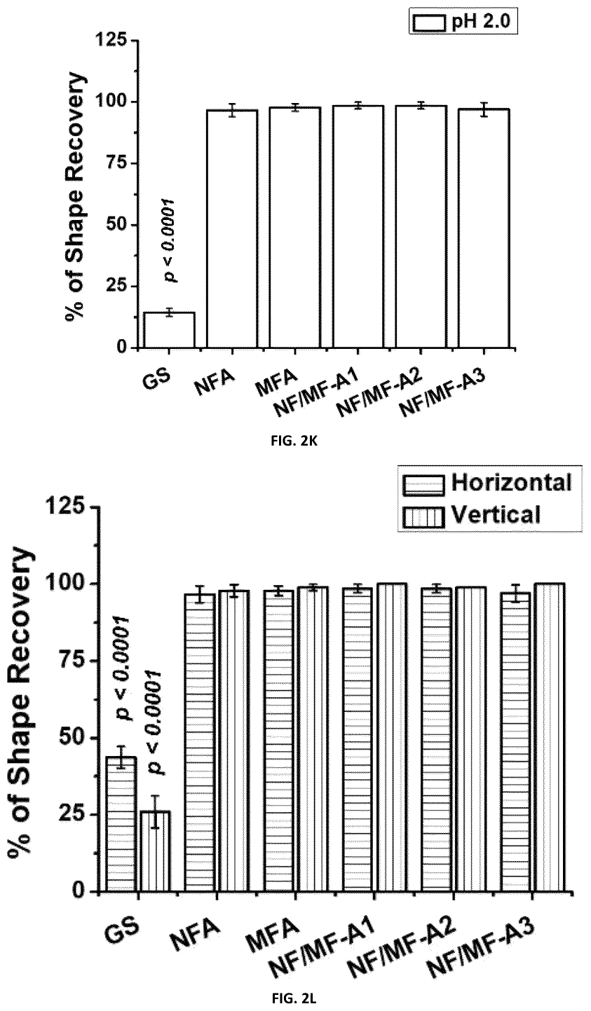

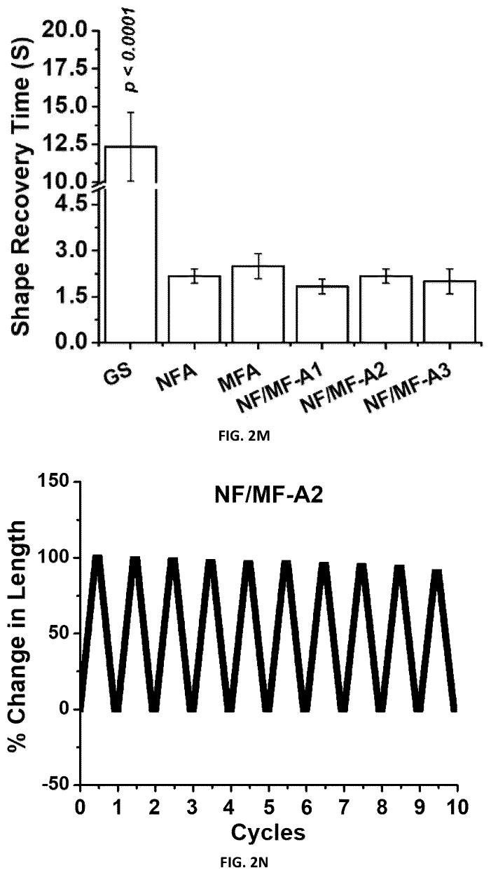

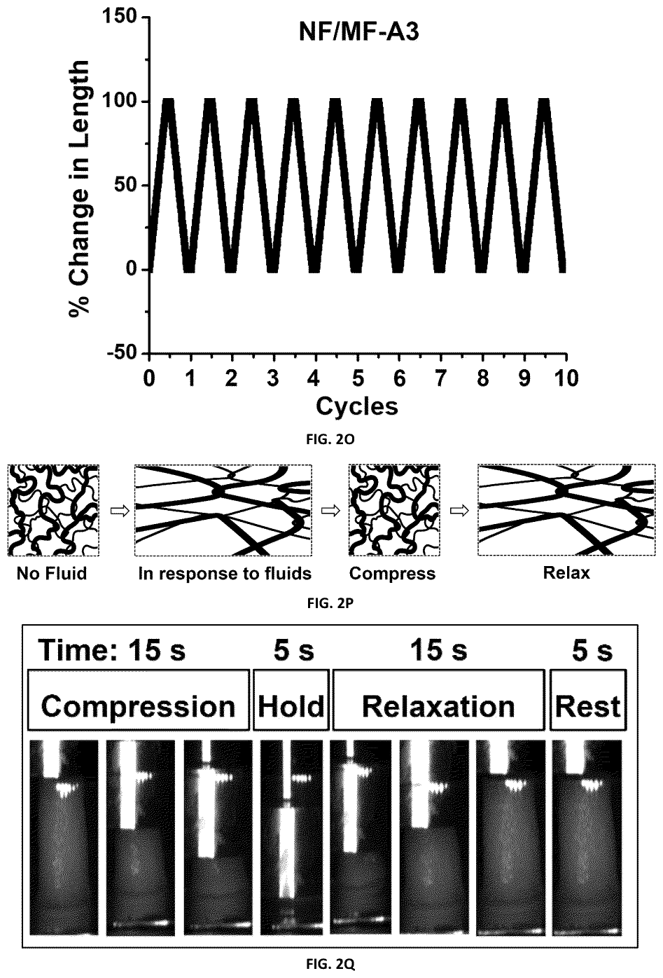

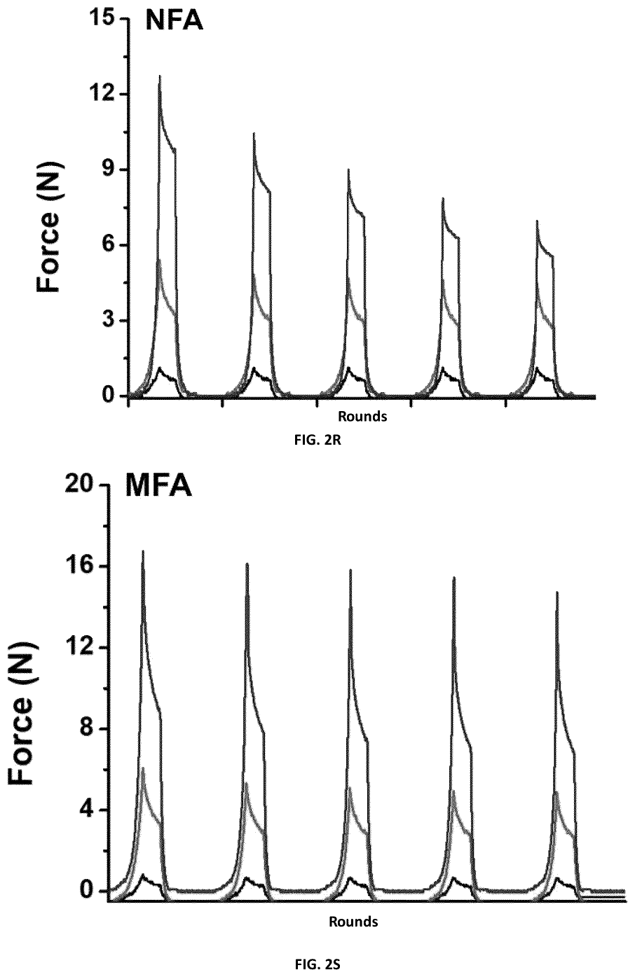

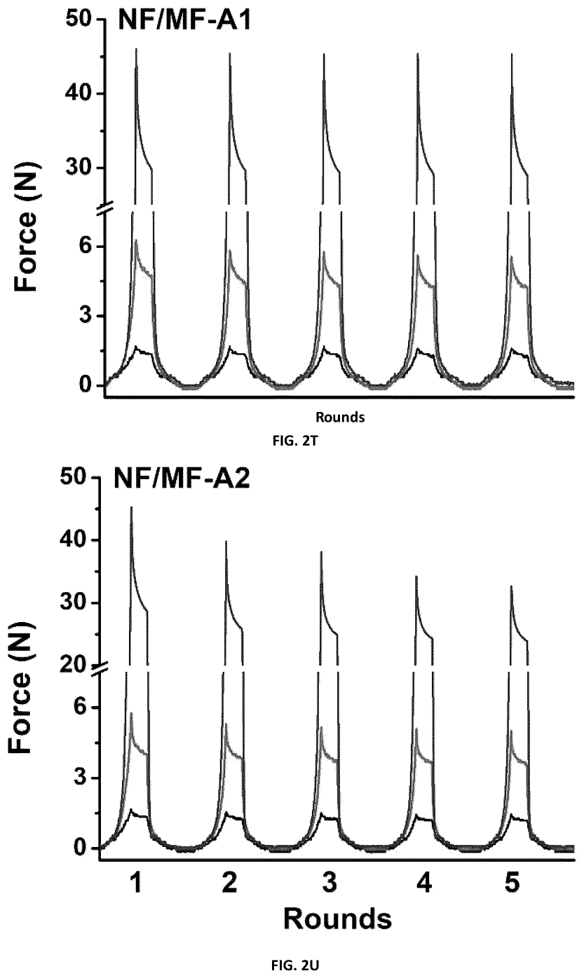

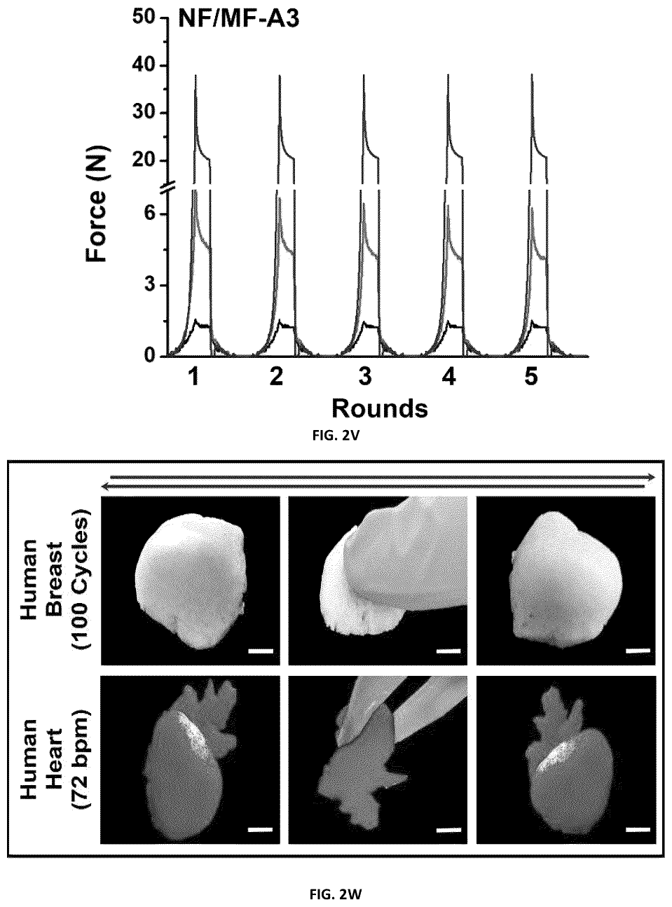

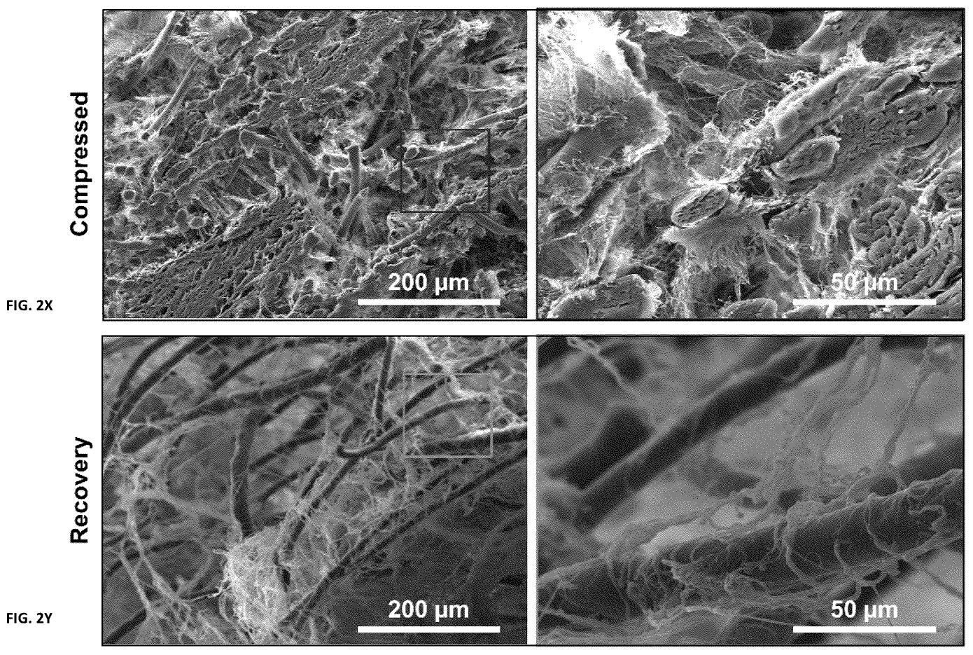

The fabricated hybrid aerogels exhibit high flexibility and excellent compression and bending resistance (FIG. 2A). In addition, hybrid aerogels can rapidly recover their shape in the solutions regardless of pH value, while gelatin sponges failed to recover their shape in acidic pH (FIGS. 2K and 2L). This finding indicates the mechanical reinforcement provided by the fibrillar networks. Their shape recovery properties at pH=2 demonstrate their application in sampling biological specimens in gastrointestinal tract in a minimally invasive manner (John, et al. (2022) Acta Biomater., 146:211-221). The shape recovery time of NF/MF-A1 (1.8±0.2 seconds) is much shorter relative to many other shape recoverable aerogels and cryogels recently developed, as previous ones often require minutes for full recovery (FIG. 2M) (Liu, et al. (2016) Advanced Materials 28:7758-7767; Du, et al. (2021) Nat. Commun., 12:4733; Liu, et al. (2019) ACS Nano 13:2927-2935; Wang, et al. (2022) Advanced Materials 34:2108325).

To better understand the mechanical properties, the mechanical strength of NFA, MFA, NF/MF-A1, NF/MF-A2, and NF/MF-A3 was examined by performing compression tests at 50%, 70%, and 90% compressive strains (FIGS. 2B and 2C). The compressive strength of aerogels increased with increasing compressive strains. Notably, NFA shows the lowest mechanical resistance among all the tested samples at all the compressive strains (FIG. 2C). The mechanical strengths of hybrid aerogels consisting of NF/MF in weight ratios of 25:75, 50:50, and 75:25 dramatically increased compared to NFA and MFA (FIGS. 2C and 2D and Tables 1 and 2). NF/MF-A1, NF/MF-A2, and NF/MF-A3 were able to endure the maximum compressive loads of 29.7±0.7 MPa, 15.1±0.1 MPa, and 12.7±0.2 MPa, whereas NFA endures a much smaller strength (1.7±0.1 MPa) under 90% compressive strain (FIG. 2C). FIG. 2D shows the excellent resilience of NF/MF-A1, which can endure 8±0.6 MPa, 11.1±1.7 MPa, and 75.2±3.6 MPa of stress under the compressive strains of 50%, 70%, and 90%. NFA shows the least resilience and poorest mechanical robustness, followed by MFA, NF/MF-A2, NF/MF-A3, and NF/MF-A1, indicating the potential application of hybrid aerogels in structural applications due to their considerable ductility.

To further demonstrate the high compression resilience of hybrid aerogels, a cyclic compression-relaxation test was performed at a rate of 1.8 mm/s (FIGS. 2E-2G, 2N, and 2O). After ten cycles at 90% strain, NFAs and MFAs show a ˜60% and ˜35% decrease in total length due to buckling and plastic deformation under large compressive loads (FIGS. 2E and 2F). Interestingly, during the compression-relaxation cycles at 90% load, compressive stress σ of NF/MF-A1 returned to the original value after unloading for each strain &. Hysteresis loop for NF/MF-A1 was not observed during the 100-cycle test (FIG. 2G). Although NF/MF-A3 shows no deformation loss in the total length, NF/MF-A2 shows a ˜7% loss under the 90% strain after ten cycles (FIGS. 2N and 2O). These results indicate that the MF network plays an important role in maintaining hybrid aerogels' high elasticity and shape-recovery. To test this, the high compressive resilience of hybrid aerogels was studied by performing another set of cyclic compression in wet conditions (FIGS. 2P-2V). Each aerogel underwent five rounds of cyclic compression with each round consisting of three cycles at three different compressive strains (FIGS. 2R-2V). NFA showed various responses to different strains, with excellent resilience at 50% and 70% and a decrease in compressive resistance at 90% (FIG. 2R). Aerogels comprising both NF and MF coated with 1% gelatin significantly showed an increase in the mechanical strength (FIG. 2T-2V). NF/MF-A1 (NF/MF weight ratio of 50:50) showed the best compression resilience and highest maximum compression stress (FIG. 2T) among all the aerogels tested, while NF/MF-A2 (NF/MF weight ratio of 75:25) showed shape recovery loss and mechanical fracture at 90% strain likely due to lower MF content (FIG. 2U). Although NF/MF-A3 (NF/MF weight ratio of 25:75) showed much lower mechanical resistance among the tested hybrid aerogels, it did not show mechanical fracture at 90% strain (FIG. 2V). These results indicate the crucial role of MFs in maintaining robust mechanical properties for hybrid aerogels. Therefore, resilience and elasticity are largely due to the presence of the NFs, while the major mechanical support is largely imparted by the MFs. The synergism of both structures unexpectedly imparts both favorable mechanical properties to the hybrid aerogels.

To further investigate the improved mechanical properties of hybrid aerogels, tearing tests were performed. The fibrous network in NFA was torn apart under the lowest tearing strength among the tested samples. The tearing strength of hybrid aerogels increased with increasing the MF content (FIG. 2H). For NF/MF-A1, the fibrous networks endured more than 6 times the tearing strength of NFA because the tension can be readily dissipated by other fibers through entanglements. To illustrate potential applications, NF/MF-A1 was fabricated with human breast and heart shapes (FIG. 2W). After a 100-cycle compression-strain test, human breast-shaped NF/MF-A1 showed no deformation or fracture. Human heart-shaped NF/MF-A1 showed high flexibility and resilience during 72 cyclic compression-strain tests per minute similar to a healthy human's heart beating rate. It was further shown that the hybrid aerogels can remain intact under strong mechanical agitation, indicating their structures are stable, which could be mainly attributed to the entanglement between NFs and MFs as well as the crosslinked gelatin component. Interestingly, NF/MF-A1 had a high fracture strength of 7448.8±228.1 Jm⁻² which is comparable to the mechanical strength of cardiac or breast tissues (FIG. 2I) (Guimaraes, et al. (2020) Nat. Rev. Mater., 5:351-370; Davenport, et al. (2016) ACS Biomater. Sci. Eng., 2:780-788; Omens, J. H. (1998) Prog. Biophys. Mol. Biol., 69:559-572; Weis, et al. (2000) Circ. Res., 87:663-669; Nagueh, et al. (2004) Circulation 110:155-162; Riedle, et al. (2018) IEEE-EMBS Conference on Biomedical Engineering and Sciences (IECBES) 171-176; Edwards, et al. (2005) J. Cardiovascular Mag. Res., 7:835-840). The mechanical properties of these hybrid aerogels are superior to previously reported aerogels known as potential candidates for tissue engineering applications in terms of their mechanical properties (FIGS. 2J and Table 1) (Qin, et al. (2015) ACS Nano 9:8933-8941; Liu, et al. (2015) Compos. Sci. Technol., 117:114-120; Wan, et al. (2017) ACS Appl. Mater. Interfaces 9:24591-24599; Jia, et al. (2019) ACS Sustain Chem. Eng., 7:12591-12600; Ding, et al. (2016) Sci. China Chem., 59:1405-1414; Gong, et al. (2019) Polymers 11:777; Guo, et al. (2013) ACS Appl. Mater. Interfaces 5:225-225; Guo, et al. (2012) ACS Appl. Mater. Interfaces 4:5422-5429). By evaluating the compressive strength, tearing strength, fracture energy, tensile modulus, and toughness of each aerogel, a weighted matrix was employed to choose the most optimized hybrid aerogel in terms of mechanical properties (Table 2). Based on the weighted T-Score table (Table 3), NF/MF-A1 was chosen for further investigations owing to the most optimal mechanical characteristics.

TABLE 1
Toughness and specific tensile modulus of hybrid
aerogels compared with other aerogels.

| | | | | Specific Tensile | |
|---|---|---|---|---|---|
| | | Porosity | Toughness | Modulus | |
| No. | Aerogels | (%) | (KJ m⁻³) | (MPa cm³ g⁻¹) | |
| 1 | NFA | 96.8 | 92.7 | 98.3 | |
| 2 | MFA | 94.3 | 398.2 | 237.1 | |
| 3 | NF/MF-A1 | 95.1 | 1961.3 | 1834.7 | |
| 4 | NF/MF-A2 | 96.4 | 800.5 | 551 | |
| 5 | NF/MF-A3 | 95.3 | 1456.2 | 1045.2 | |
| 6 | CNA-91 | 91 | 212 | 136.4 | (A) |
| 7 | CNA -87 | 87 | 4.6 | 205.6 | (A) |
| 8 | CNA-83 | 83 | 523 | 321.9 | (A) |
| 9 | CNA-76 | 76 | 1050.6 | 625.3 | (A) |
| 10 | BC | — | 207.4 | 45.2 | (B) |
| 11 | BC/PEDOT/SWCNT | 76.8 | 22.7 | 240 | (C) |
| 12 | Chitin | 87 | 480.7 | 473.5 | (D) |
| 13 | Chitosan | 83.7 | 147.6 | 550.6 | (E) |
| 14 | PI | 80.3 | 362.3 | 558.3 | (F) |
| 15 | PI/CNT | 59.8 | 152.8 | 149.5 | (G) |
| 16 | PI/Graphene | 98.9 | 9 | 173.8 | (H) |

(A): He, et al. (2022) Nat. Commun., 13: 4242.
(B): Wan, et al. (2017) ACS Appl. Mater. Interfaces 9: 24591-24599.
(C): Jia, et al. (2019) ACS Sustain Chem Eng., 7: 12591-12600.
(D): Ding, et al. (2016) Sci. China Chem., 59: 1405-1414.
(E): Gong, et al. (2019) Polymers 11: 777.
(F): Guo, et al. (2012) ACS Appl. Mater. Interfaces 4: 5422-5429;
(G): Liu, et al. (2015) Compos. Sci. Technol., 117: 114-120.
(H): Qin, et al. (2015) ACS Nano 9: 8933-8941.

TABLE 2
Summary of mechanical properties.

| Criteria | Weight | NFA | MFA | NF/MF-A1 | NF/MF-A2 | NF/MF-A3 |
|---|---|---|---|---|---|---|
| Compressive | 0.13 | 1.802 | 6.472 | 28.758 | 15.311 | 23.715 |
| Strength | | | | | | |
| (MPA) | | | | | | |
| Tearing | 0.18 | 2.466 | 4.678 | 14.816 | 9.837 | 12.167 |
| Strength (N) | | | | | | |
| Fracture | 0.14 | 1.233 | 2.339 | 7.408 | 4.918 | 6.083 |
| Energy | | | | | | |
| (kJ/m²) | | | | | | |
| Youngs | 0.15 | 3.57 | 15.327 | 75.482 | 30.807 | 56.044 |
| modulus | | | | | | |
| (MPA) | | | | | | |
| Toughness | 0.3 | 92.782 | 398.263 | 1961.367 | 800.509 | 1456.292 |
| (KJ/m³) | | | | | | |
| Density | 0.1 | 0.036 | 0.064 | 0.041 | 0.059 | 0.053 |
| (g/cm³) | | | | | | |

| | | | | | |
|---|---|---|---|---|---|
| Total | 101.889 | 427.143 | 2087.872 | 861.441 | 1554.354 |
| Weighted Average | 29.225 | 123.795 | 607.179 | 249.229 | 451.424 |

TABLE 3
Weighted T-Score table.

| Criteria | Weight | NFA | MFA | NF/MF-A1 | NF/MF-A2 | NF/MF-A3 |
|---|---|---|---|---|---|---|
| Compressive | 0.13 | 51.11 | 62.42 | 64.112 | 57.54 | 63.88 |
| Strength | | | | | | |
| (MPa) | | | | | | |
| Tearing | 0.18 | 59.27 | 58.89 | 63.92 | 58.333 | 60.52 |
| Strength (N) | | | | | | |
| Fracture | 0.14 | 53.57 | 58.57 | 62.49 | 52.5 | 58.57 |
| Energy | | | | | | |
| (kJ/m²) | | | | | | |
| Young's | 0.15 | 53.33 | 55.66 | 60.00 | 56.54 | 59.62 |
| modulus | | | | | | |
| (MPa) | | | | | | |
| Toughness | 0.3 | 58.88 | 62.76 | 78.42 | 63.56 | 68.90 |
| (KJ/m³) | | | | | | |
| Density | 0.1 | 58.06 | 58.64 | 66.14 | 59.86 | 63.02 |
| (g/cm³) | | | | | | |

| | | | | | |
|---|---|---|---|---|---|
| Total | 334.25 | 356.94 | 395.08 | 348.33 | 374.00 |
| Weighted Average | 56.28 | 59.95 | 67.72 | 60.96 | 63.31 |

To understand the mechanism of shape-recovery and super elasticity of NF/MF-A1, SEM imaging was performed under 90% compressive strain and after shape recovery (FIG. 2X-2Z). When compressing wet hybrid aerogels, the absorbed water squeezed out due to the deformation of pores, exhibiting a compact/dense micro- and nano-fibrillar structure (FIG. 2Y). The GA crosslinked gelatin and entanglement between fibers can form networks and bridge the fibrillar junctions, thus enabling stress transmission throughout aerogels and preventing the collapse of fibrillar network. Under 90% compressive strain, the hybrid aerogel forms a compressed pellet storing a large amount of elastic potential energy without collapsing or force dissipation. When the compressed pellet in contact with water, it absorbs a large amount of water, and regains its original shape quickly and exerts force due to the release of stored elastic potential energy (FIGS. 2Y and 2Z). The shaped-recovered aerogels show similar structures to the ones prior to compression (FIGS. 2G and 2Z).

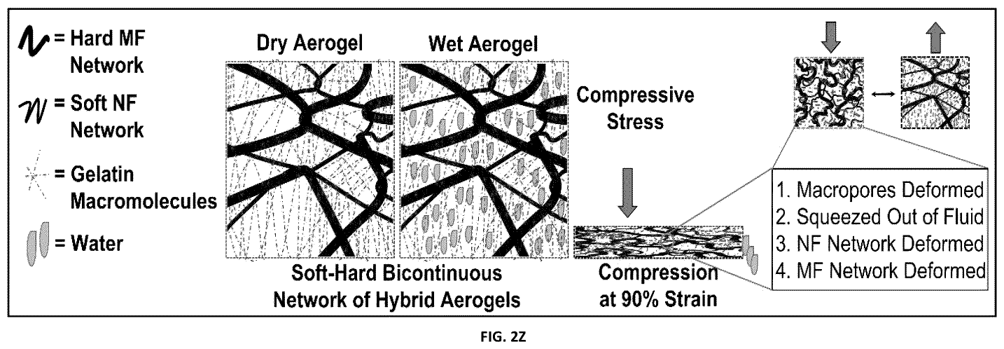

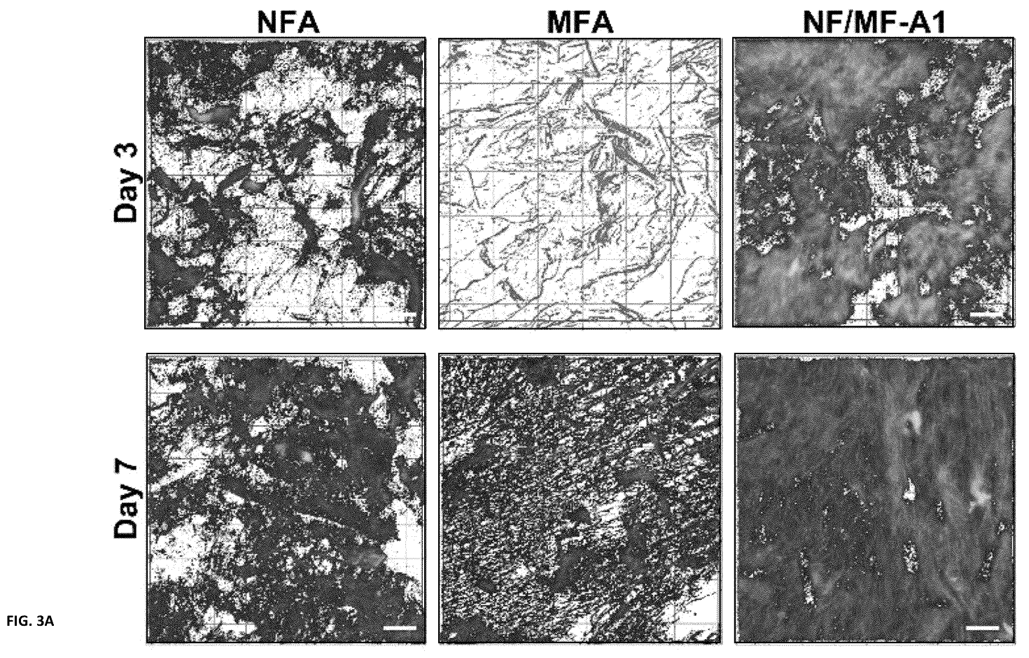

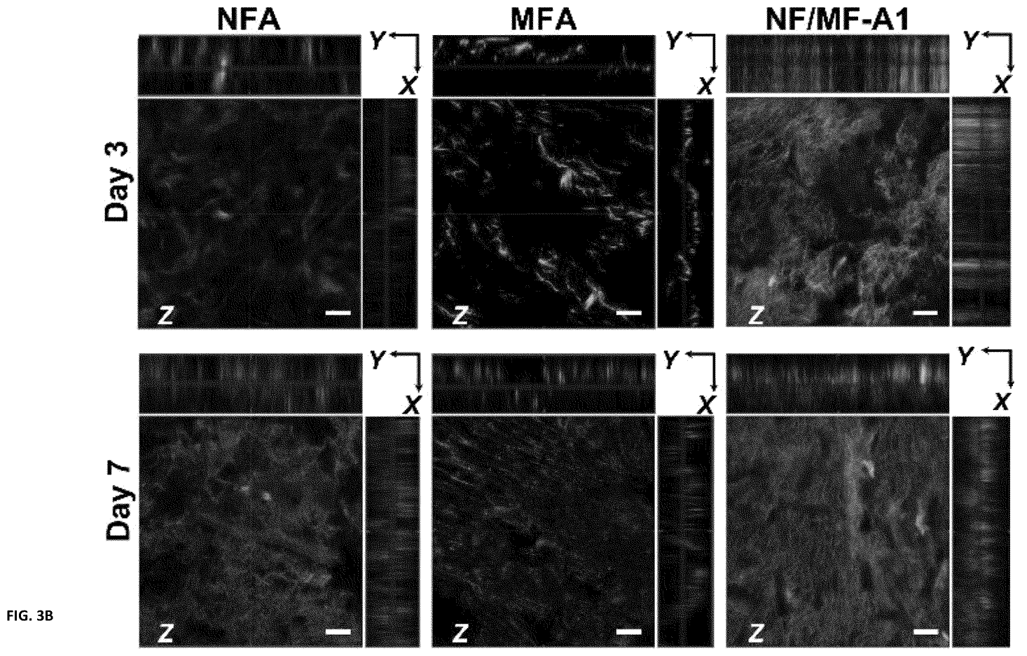

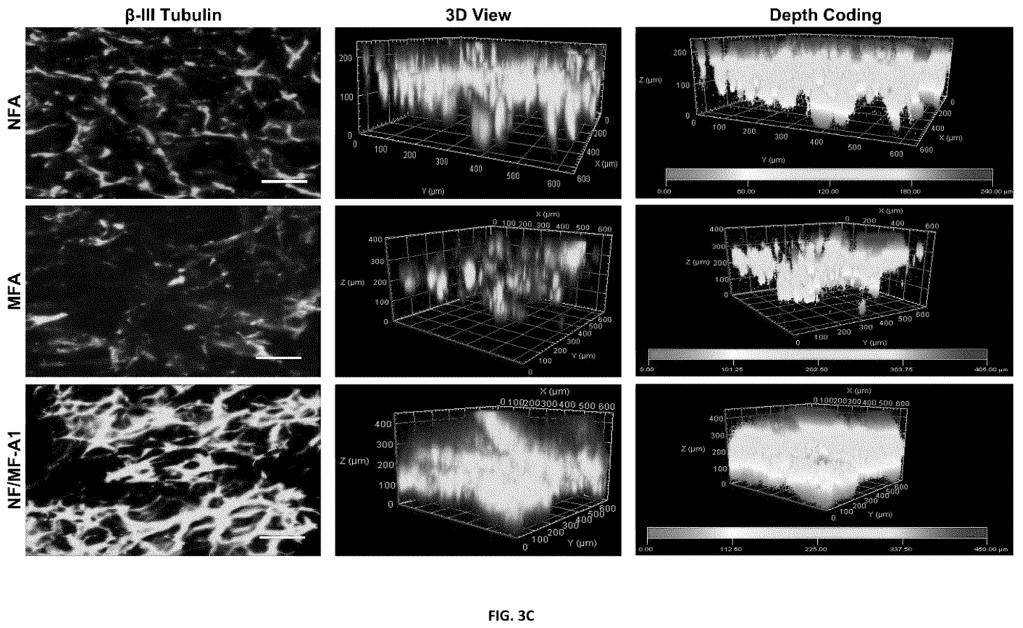

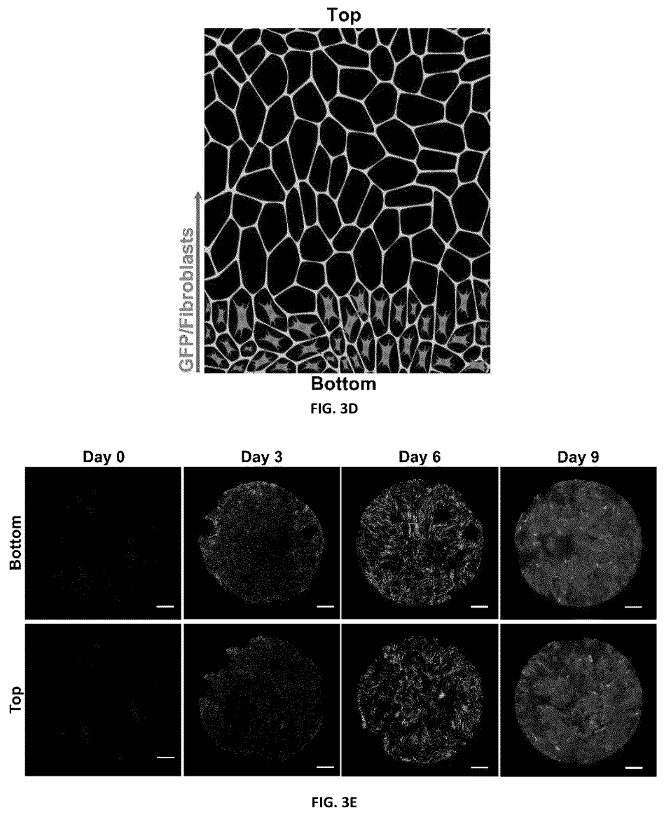

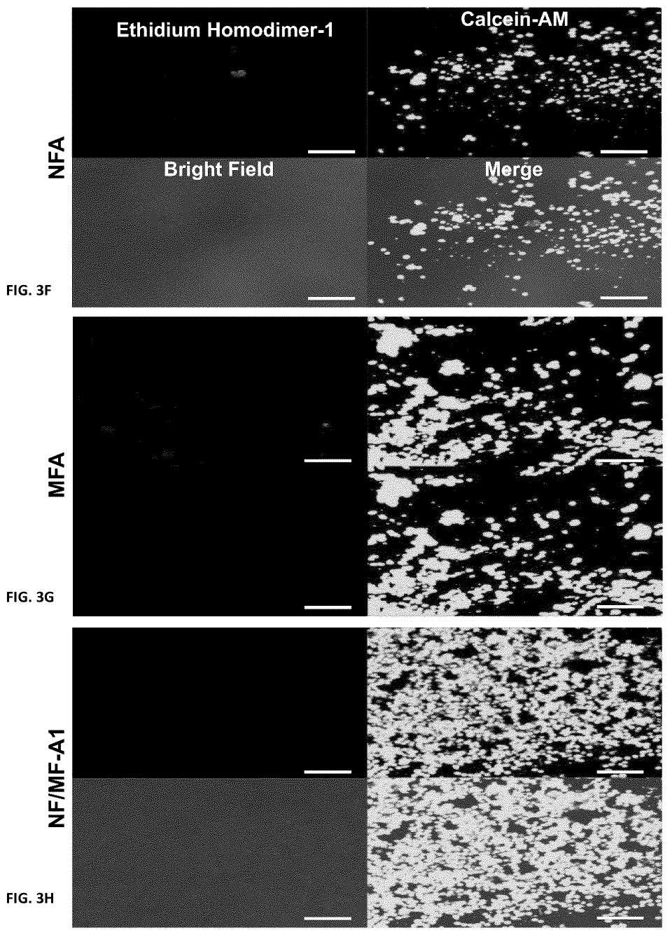

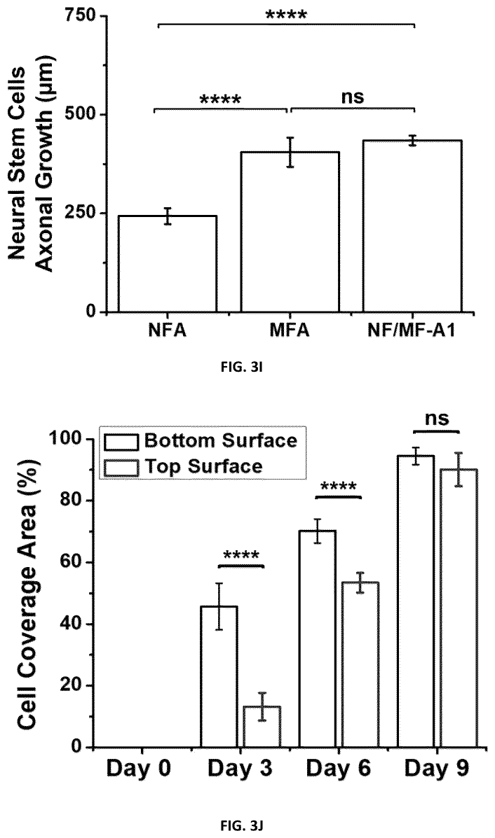

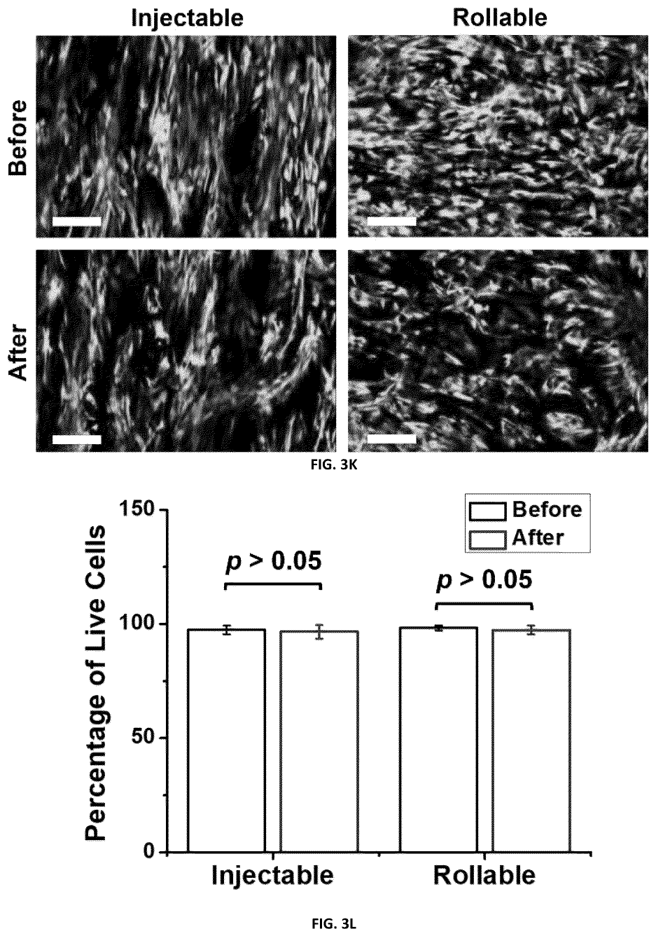

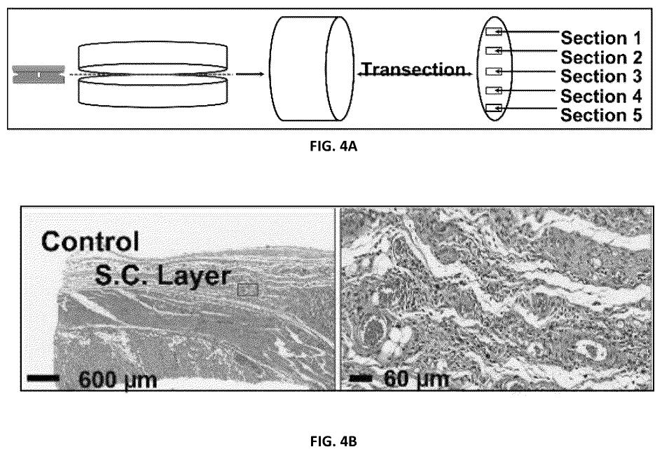

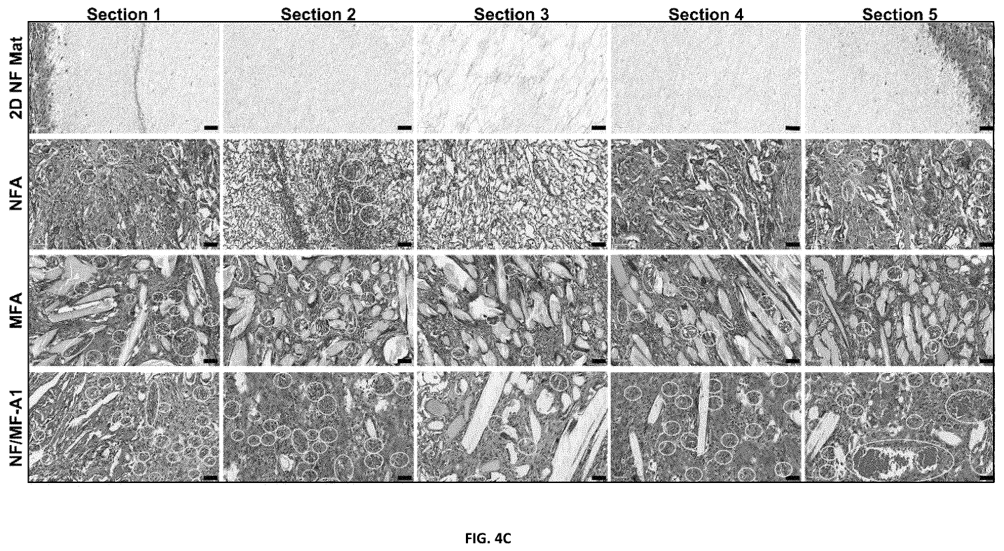

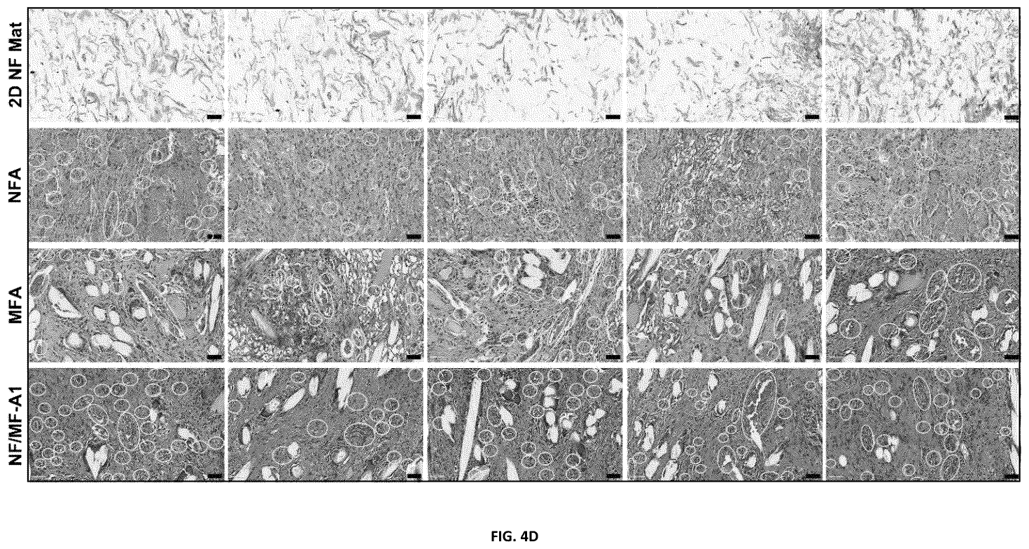

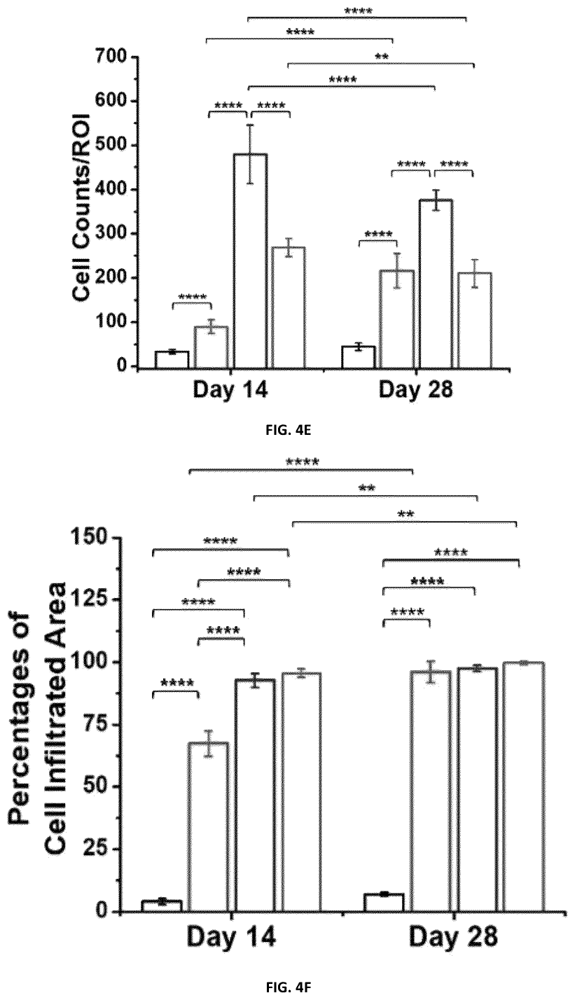

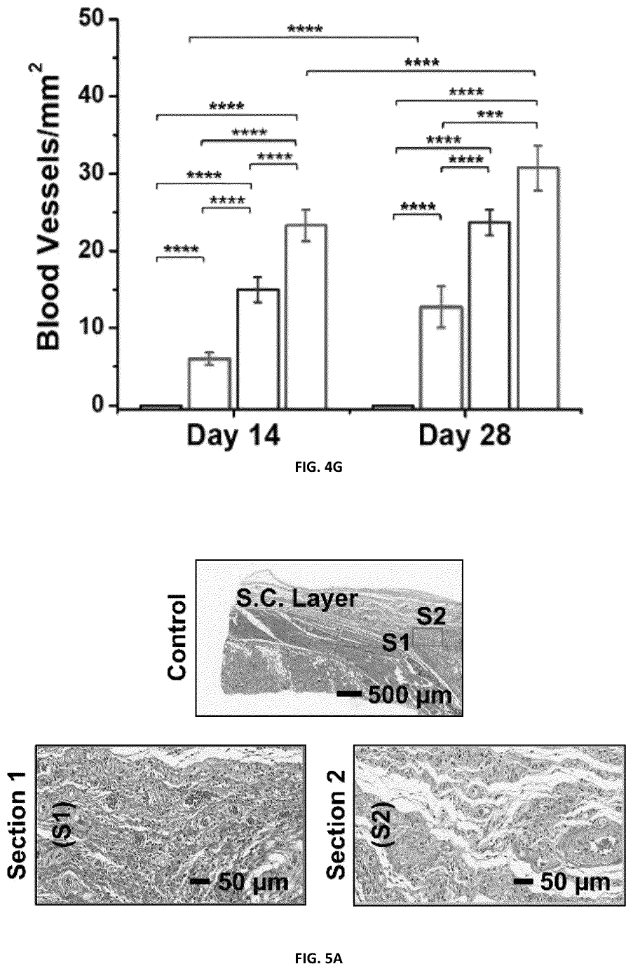

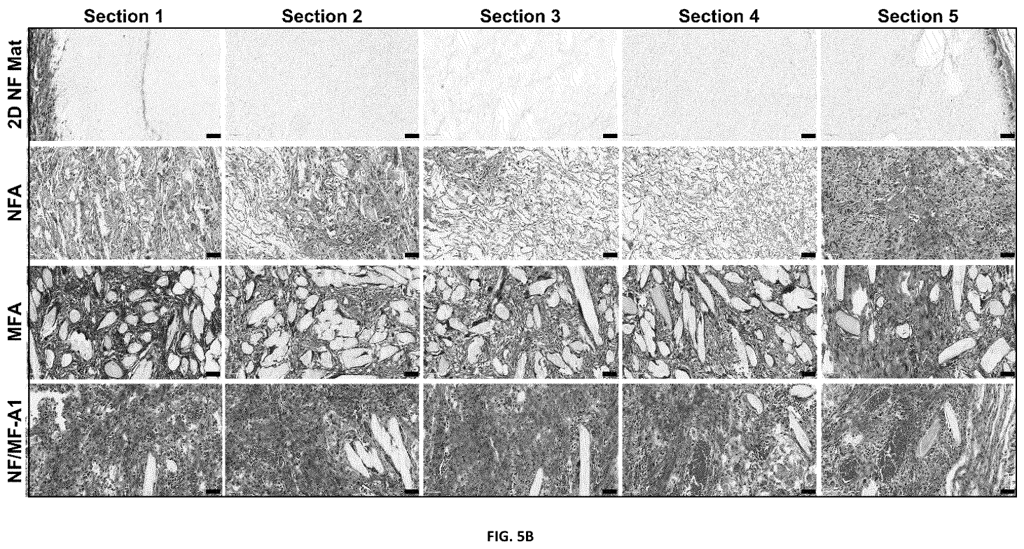

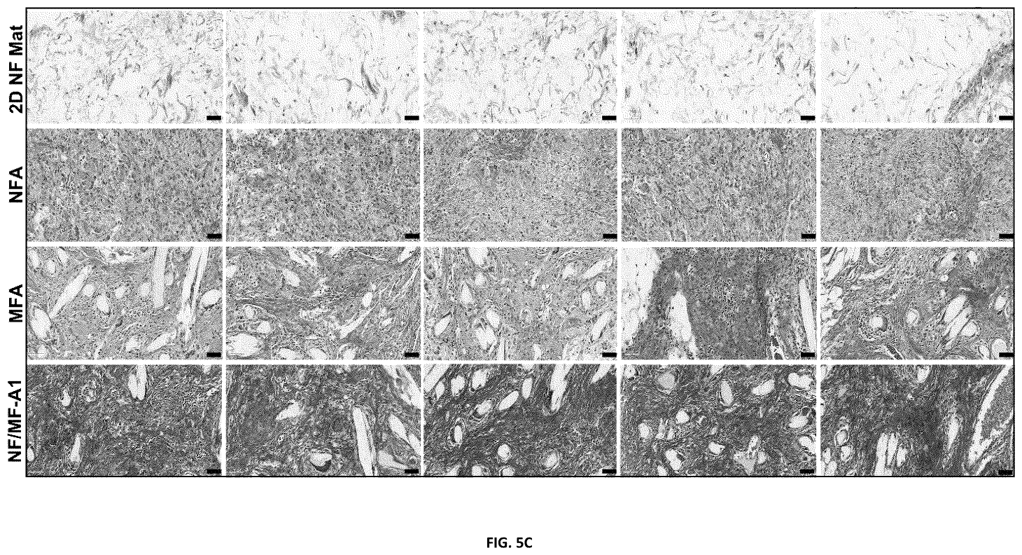

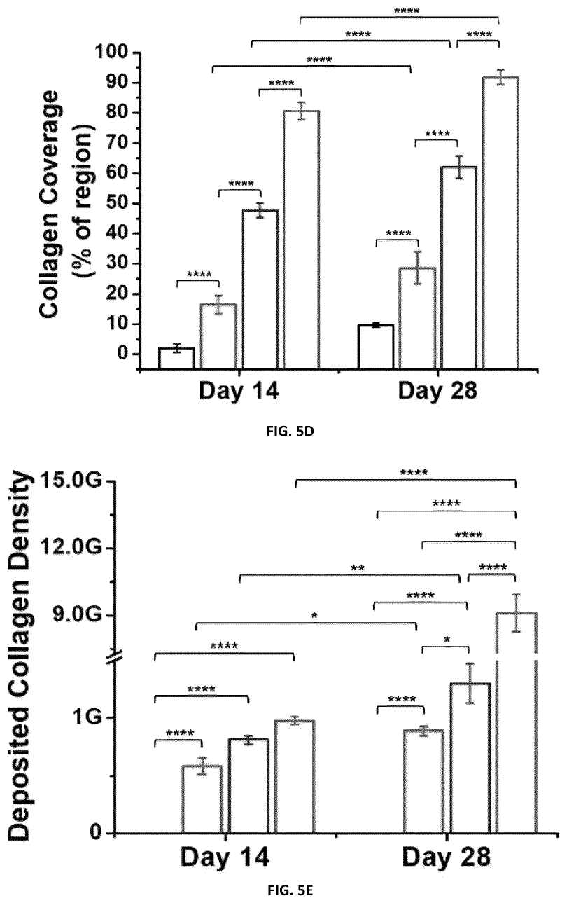

The hybrid aerogels composed of entangled NFs and MFs can recapitulate the natural ECM structure and regulate cell responses (Qian, et al. (2018) Macromol. Rapid Commun., 39:1700724). In addition, the highly interconnected porous structure can facilitate cell infiltration and the exchange of nutrition and waste during cell culture. To test this, HaCaT cells (immortalized human keratinocytes) were cultured on aerogels with different ratios of NFs and MFs (FIGS. 3F-3H). After 36 hours of culture, the live/dead staining of HaCaT cells cultured on either NFA, MFA, or NF/MF-A1 was performed. HaCaT cells showed higher proliferation and more uniform distribution on NF/MF-A1 when compared to NFA or MFA, indicating that NF/MF-A1 provides a superior environment for cell infiltration and proliferation, which is imparted by both the NFs and MFs (FIGS. 3F-3H). Due to the fiber size in tens of microns, the pores in MFA were large enough for cell infiltration when seeding cells. However, the seeded cells only interacted with MFs which may be less optimal for cell proliferation relative to NFs. In contrast, because of the fiber size in several hundreds of nanometers, the pore size in NFA was small and thus NFA was not easily penetrated by cells and more seeded cells may be located on the surface layer of NFA. NF/MF-A1 had large pores and NF networks, which would allow cell infiltration during cell seeding, and provide a biomimetic environment for promotion of cell growth. GFP-labeled dermal fibroblasts were also cultured on different aerogels. The GFP-labeled dermal fibroblasts showed a similar trend of sustained and evenly distributed proliferation. More dermal fibroblasts were seen on NF/MF-A1 at days 3 and 7 after seeding compared to NFA and MFA (FIG. 3A). Importantly, the dermal fibroblasts proliferate throughout the whole hybrid aerogels (FIG. 3B), indicating their application in preparing 3D tissue constructs, rather than restricting cellular migration to a planar environment.

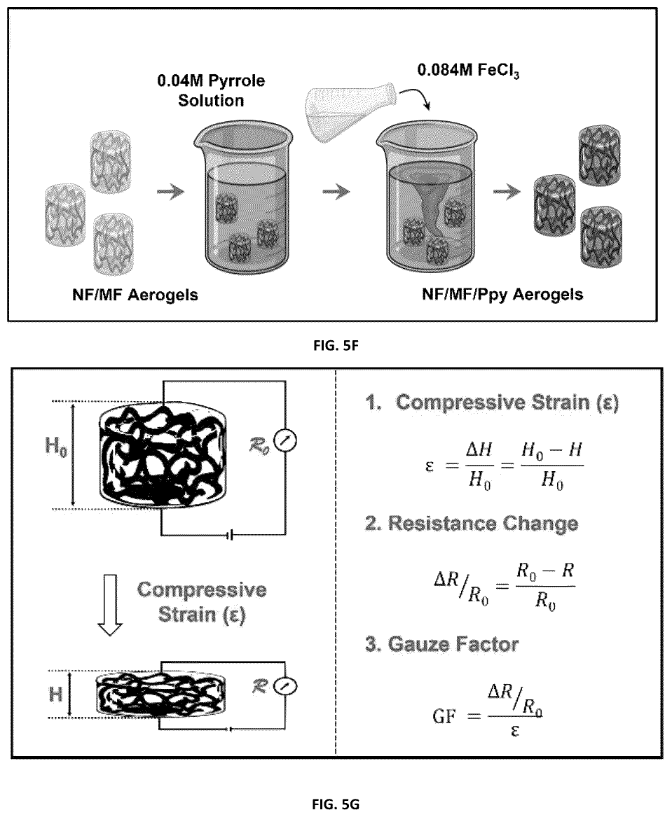

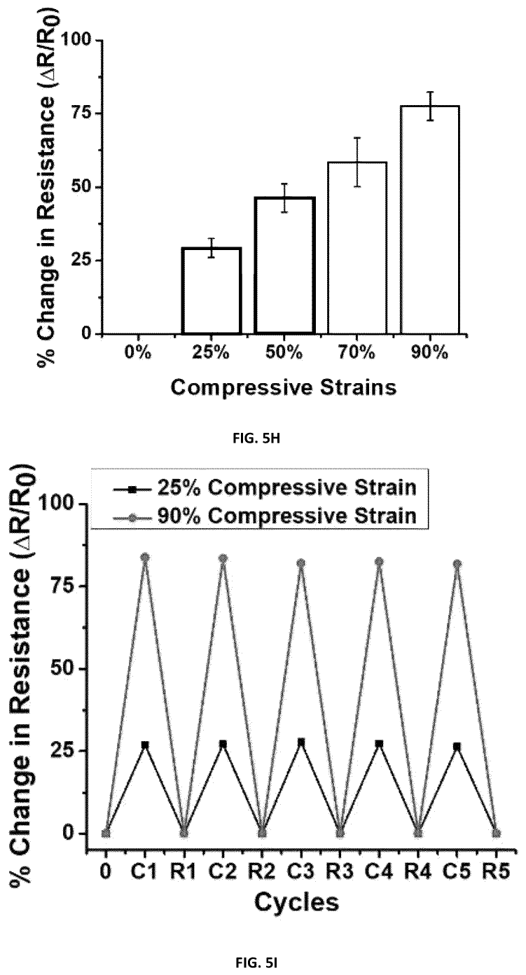

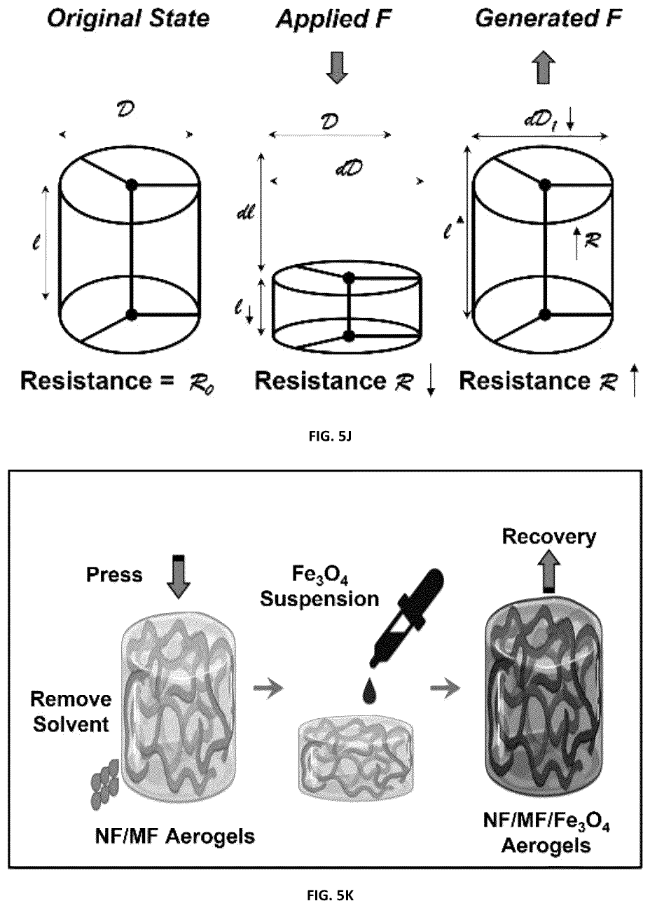

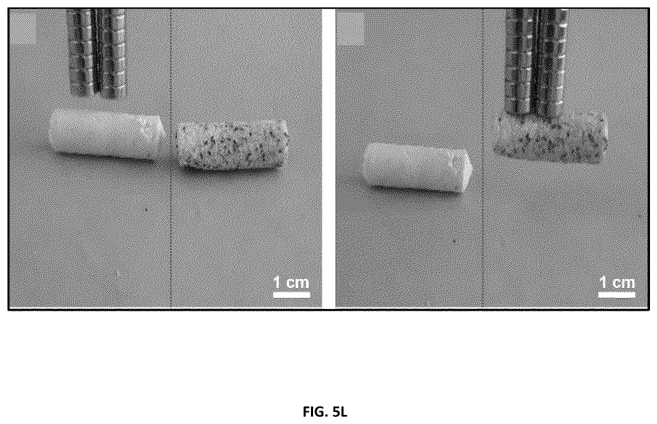

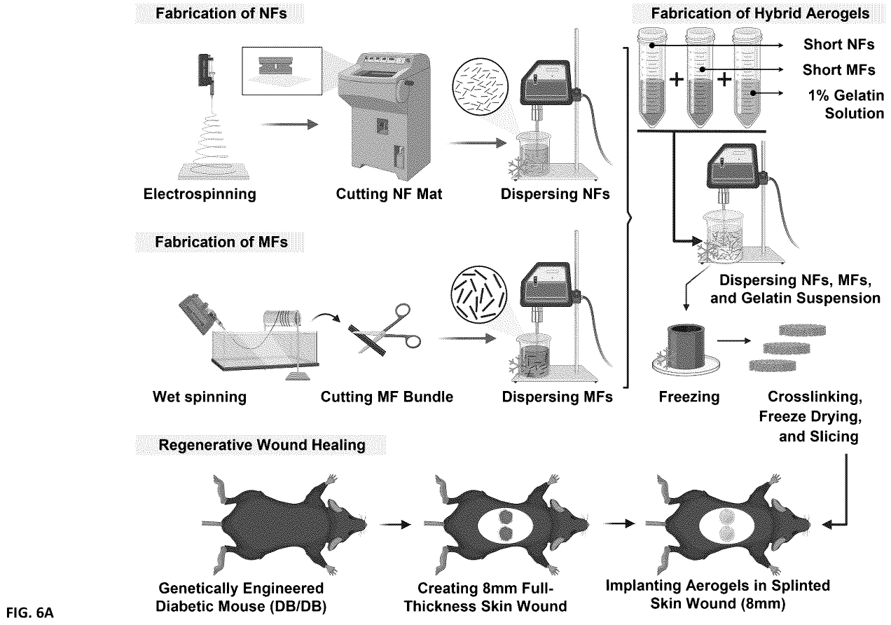

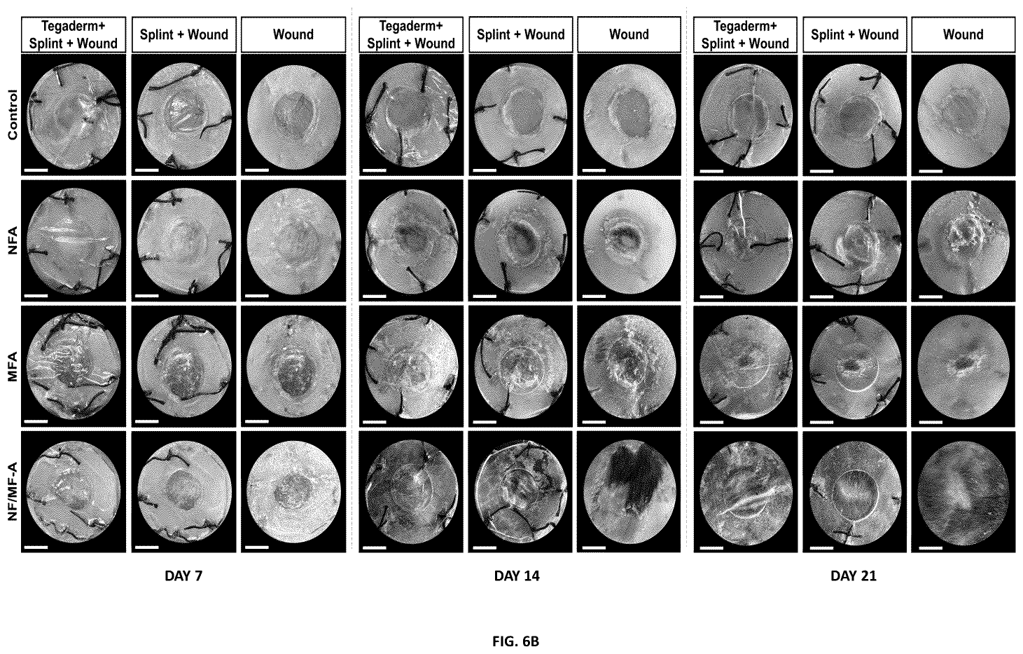

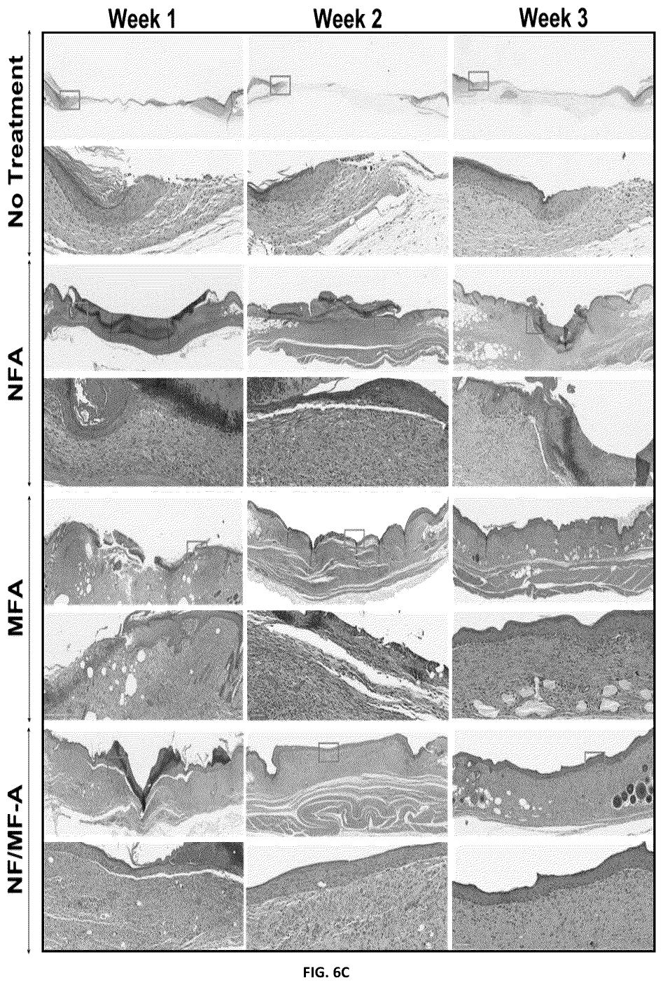

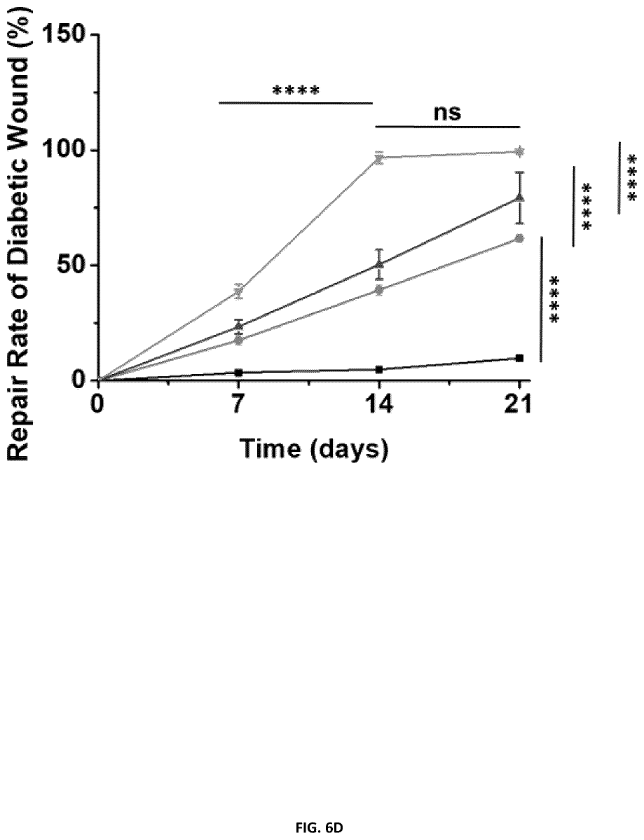

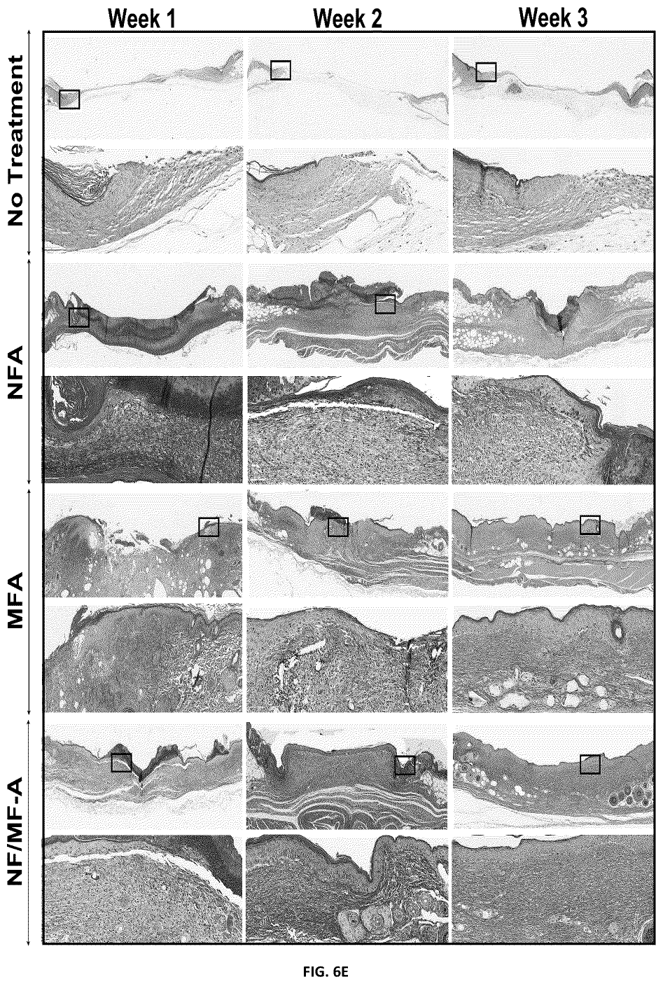

In addition to skin cells, the differentiation of seeded human neural precursor/stem cells were investigated on NFA, MFA, and NF/MF-A1 and neurite outgrowth were found mainly occurred on the top surface of NFA, indicating that the initial cell seeding into NFA failed, whereas neurite outgrowth on MFA was significantly deeper, indicating that the cells were able to infiltrate into the deeper location during cell seeding attributed to the larger pores of MFA (FIG. 3C). Hybrid aerogels not only promoted stem cell infiltration but also enhanced neurite outgrowth, which could be due to the large pores and NF networks (FIG. 31). The resultant 3D neural networks on NF/MF-A1 may be useful for modeling neural development and neurological disorders, and screening nerve toxic drugs (Chen, et al. (2020) Appl. Phys. Rev., 7:021406).

To further examine the function of NF/MF-A1 in regulating cell proliferation and migration, GFP-fibroblasts were first seeded on petri dishes coated with GelMA. Then, NF/MF-A1 (diameters×height: 8 mm×2 mm) was placed on the top of cells. The migration of cells from bottom to top was examined by confocal laser scanning microscopy (CLSM) imaging at pre-determined time points (FIG. 3D). FIGS. 3E and 3J show that the area with green fluorescence on the top of the aerogels gradually increased over time, indicating the migration and proliferation of the GFP-fibroblasts from the bottom to the top surface.

The highly flexible, shape-recoverable properties of hybrid aerogels and the ability for cells to migrate and proliferate rapidly within them indicates their potential applications in delivering functional 3D tissue constructs in a minimally invasive manner (Zhu, et al. (2021) Nat. Commun., 12:1412; Chen, et al. (2021) ACS Biomater. Sci. Eng., 7:2204-2211). To demonstrate this, GFP-labeled dermal fibroblasts were seeded on NF/MF-A1 with different sizes (diameters×height: 8 mm×2 mm, 10 mm×2 mm, 12 mm×2 mm, and 7 mm×10 mm). After 14 days of culture, the formed tissue constructs were loaded into an applicator with a 1.6 mm inner diameter cannula and injected into cell culture dishes. Such constructs can be delivered through the cannula with a much smaller diameter than the aerogels, which has not been demonstrated for any other type of aerogels. For minimally invasive delivery, it is not necessary to either compress, roll, or fold hybrid aerogel-based tissue constructs inside the applicator unlike previously reported scaffolds (Huynh, K. (2017) Nat. Rev. Cardiol., 14:568-568; Montgomery, et al. (2017) Nat. Mater., 16:1038-1046; Beduer, et al. (2015) Adv. Healthc. Mater., 4:301-312). The uncompressed aerogels were able to deform and pass through the cannula likely because of their ultra-flexibility and the applied shear stress during injection. In addition, the composite aerogel created by decellularizing dermal fibroblasts-seeded NF/MF-A1 can be rolled up and regain its shape instantaneously after being delivered through a cannula. This aerogel showed high elastic modulus and resilience with outstanding shape-memory properties after delivery in a minimally invasive manner. It is worth noting that no significant variation in the cell viability of GFP-labeled dermal fibroblasts was observed on the hybrid aerogels before and after the delivery (FIGS. 3K and 3L).

To investigate the potential in tissue regeneration, 2D NF mats, NFA, MFA, and NF/MF-A1 were subcutaneously implanted as acellular scaffolds to four supraspinal sites in the dorsum of rats and acquired biopsied at 14 and 28 days. The post-surgical images indicated no severe adverse tissue responses regarding necrosis, inflammation, or infection related to the implanted materials at any time point. More host cell infiltration was seen after 14 days of implantation in NF/MF-A1 and MFA than in 2D NF mats (p<0.0001) and NFA (p<0.0001) (FIGS. 4A-4E). This rapid cell penetration was most likely attributes to the interconnected large pores in NF/MF-A1 and MFA. Such a structure allowed the host cells to migrate from the surrounding tissues to the implants and form new tissues. After 28 days of implantation, host cells completely penetrated the NFA, MFA, and NF/MF-A1 forming newly vascularized tissues. Within the same periods, due to the dense structure and small pore size, cells were mainly located on the surface layer of 2D NF mats and failed to penetrate, which was in line with other studies (Rnjak-Kovacina, et al. (2011) Tissue Eng. Part B Rev., 17:365-372; Khorshidi, et al. (2016) J. Tissue Eng. Regen. Med., 10:715-738; Chen, et al. (2020) J. Mater. Chem. B, 8:3733-3746; Daracinejad, et al. (2021) Front. Bioeng. Biotechnol., 9:641371; Maksoud, et al. (2022) J. Mater. Chem. B, 10:8111-8165). The areas of cell infiltration and number of new blood vessels formed in each explant were quantified at different time points (FIGS. 4F-4G). The mean cell infiltrated areas for the 2D NF mats, NFA, MFA, and NF/MF-A were 4.1±1.1%, 68.8±5.5%, 92.3±2.8%, 95.7±1.7% after 14 days of implantation (FIG. 4F). The high percentage of cell infiltrated area in NF/MF-A1 (p<0.0001) within 14 days indicated their great potential applications in tissue regeneration and wound healing. In contrast, the cell infiltrated area reached 96.2±4.3% for NFA after 28 days of implantation. As expected, 2D NF mats showed marginal cell infiltration at both time points. Interestingly, the number of new blood vessels inside the NF/MF-A1 was significantly higher than NFA (p<0.0001) and MFA (p<0.01) on day 14 and 28 (FIG. 4G). The blood vessels were evenly distributed throughout the entire newly formed tissues within hybrid aerogels, which is comparable with native subcutaneous tissues (FIGS. 4C and 4D).

Moreover, Masson's trichrome staining showed enhanced ECM collagen production and neovascularization in the implanted NF/MF-A among all the tested materials at days 14 (p<0.0001) and 28 (p<0.0001) (FIGS. 5A-5C). The area of collagen deposited increased over time in all materials (FIG. 5D). The materials with the highest to lowest amount of collagen deposition were NF/MF-A1, MFA, NFA, and 2D NF mats (FIG. 5E). The hybrid aerogels had significantly more collagen deposition than NFA (p<0.0001) or MFA (p<0.0001), especially at day 28. Without being bound by theory, this could be due to two distinctly different mechanisms of action. One is that the large-sized microfibers could activate mechanoreceptors, causing an increase in cell proliferation and ECM synthesis (Varani, et al. (2004) J. Investigative Dermat., 122:1471-1479). The other is that the short nanofibers could be undergone phagocytosis, which may lead to a subclinical inflammatory response, resulting in macrophage-mediated neocollagenesis (Ray, et al. (2020) J. Funct. Biomater., 11:51). The deposited collagen was homogenously distributed throughout the entire NF/MF-A1, which is akin to the native subcutaneous tissue (FIGS. 5A-5C).

The cross-sectional morphologies of each explant were examined at days 14 and 28 μsing SEM. The cross-section of 2D NF mat explant showed the dense NFs, indicating that cells failed to penetrate this material due to the compact structure and small pore size. In stark contrast, the cross-sections of MFA and NF/MF-A1 explants showed the deposited ECM, red blood cells, and MFs. This result agreed well with histology results of fast cell infiltration, new blood vessel formation, and ECM deposition through the entire NF/MF-A1 at early time points. Notably, the ECM fibers deposited appear aligned and highly oriented, indicating the regenerative mechanism results in non-fibrotic neotissue formation. NFs were not visible in the cross-sections of NFA and NF/MF-A1 explants, which was in line with the histological observations (FIGS. 4 and 5), indicating NFs could be degraded or integrated with newly formed tissue and deposited ECM. However, the “void areas” resulting from the presence of microfibers are observable in hematoxylin and eosin (H&E) and MT staining, a finding corroborated by SEM analysis. The hybrid aerogels can also be incorporated with biological cues to further promote host cell recruitment, ECM production, and angiogenesis.

Finally, hybrid aerogels can be modified with desired functions. Hybrid aerogels were coated with polypyrrole (NF/MF/Ppy), exhibiting strain-responsive piezoresistive pressure sensing characteristics (FIG. 5F-5J). The resistance varied with the compressive strains and returned to its original value in each cycle upon the release of compressive stress (FIGS. 5H and 5I). Under the compressive stress, the length of the porous structure decreased, the electron transport path decreased, and the cross-sectional area for electrical conduction increased, likely resulting in a decrease in electrical resistance (FIG. 5J) (Chen, et al. (2020) Appl. Phys. Rev., 7:021406; Lu, et al. (2014) Sci. Rep., 4:5792). Given the excellent shape-recovery property and piezoresistive response in the dry state (FIG. 5F-5J), NF/MF/Ppy could be used for pressure sensing and electrical stimulation (Song, et al. (2022) Nanomicro. Lett., 14:152; Xie, et al. (2009) Adv. Funct. Mater., 19:2312-2318; Liang, et al. (2020) Bioelectricity 2:101-119; Song, et al. (2021) Biomaterials 275:120982). In addition, the movement of hybrid aerogels functionalized with Fc304 nanoparticles (NF/MF/Fc304) can be casily manipulated by a magnet, indicating excellent magnetic responsive properties in both dry and wet states (FIG. 5K-5L). Such magnetic field responsive hybrid aerogels could be used as actuators or magnetic resonance imaging (MRI)-visible scaffolds for tissue regeneration.

In summary, a new class of biomimetic 3D hybrid aerogels fabricated by combining electrospinning, wet spinning, freeze casting, and crosslinking has been demonstrated. The chemical crosslinking and physical entanglements of fibrillar networks render hybrid aerogels high mechanical strength and outstanding resilience. Hybrid aerogels exhibited a highly porous fibrillar structure with high resilience to stress and rapid shape-recovery property. NF/MF-A1 can fully regain its shape in less than 2 seconds by exerting 29.6±0.62 MPa of pressure. The robust compression strength of hybrid aerogels caters to the essential equilibrium between strength and flexibility crucial for soft tissue regeneration, particularly in dynamic tissues like the heart and breast, necessitating superior fracture energy and mechanical robustness. Moreover, the hybrid aerogels, characterized by networks of fibers of dual-scale dimensions, serve as a conducive environment for cell proliferation, migration, and neurite outgrowth in vitro and present an ideal matrix in vivo for fostering host cell infiltration and ECM production. Additionally, the early development of neovascularization facilitates efficient substance exchange, especially in large tissue repair situations. Furthermore, the functionalization of hybrid aerogels has been demonstrated with strain-responsive sensing and magnetic-responsive properties. The hybrid aerogels developed herein can be used for many applications including regeneration of various types of tissues, pressure sensing, actuator, and imaging.

### Example 2

FIG. 6A provides an illustration of the short nanofibers and microfibers fabrication processes and the fabrication steps involved in making hybrid aerogels. FIG. 6A also depicts the utilization of hybrid aerogels in regenerative wound healing of DB/DB mice (e.g., from Charles River Laboratories) and an overview of the surgical procedure.

FIG. 6B provides photographic documentation of the post-treatment progression of diabetic wounds in DB/DB mice (n=8) at intervals of 7-, 14-, and 21-days post-surgery (left to right, respectively). Two full-thickness 8 mm splinted skin wounds were surgically created on the dorsal area of DB/DB mice. Subsequently, nanofiber aerogels (NFA), composed of short nanofibers and 2% gelatin; microfiber aerogels (MFA), comprising short microfibers and 2% gelatin; and hybrid aerogels (NF/MF-A), comprising of a 50/50 w/w ratio of NF and MF, coated with 2% gelatin, were implanted. Wound contraction was prevented using suture splints. Following aerogel implantation, wounds were covered with Tegaderm™ for protection. Mice receiving no treatment after surgery served as control group for the experiment.

FIG. 6C provides H&E staining images depicting the histological analysis of healed wounds at days 7, 14, and 21. In untreated mice, there was no observed healing even after 21 days. The NFA-treated group did not exhibit wound healing within 21 days. Conversely, the MFA-treated group displayed extensive wound healing and NF/MF-A treated group displayed complete wound healing within 14 days.

FIG. 6D provides a graph of the percentage of wound healing observed in in vivo studies conducted over 21 days. The data are presented as mean±standard deviation (SD), with a sample size of n=8. Statistical analysis was performed using one-way ANOVA followed by Tukey's post hoc test. As seen in FIG. 6D, the repair rate of the diabetic wound in NF/MF-A treated was 100% by day 14. In contrast, the repair rate of the diabetic wound in NFA-treated and MFA-treated groups failed to reach 100% even by day 21.

FIG. 6E provides Masson's trichrome (MTC) staining results of healed wounds over 21 days. No collagen deposition was observed in the untreated group, consistent with the absence of wound healing, as corroborated by H&E findings (FIGS. 6C and 6D). In contrast, all aerogel-treated groups exhibited collagen deposition in the healed area along with early formation of blood vessels. Notably, NF/MF-A treatment resulted in complete remodeling of the healed skin, characterized by sufficient blood vessel formation and extracellular matrix remodeling.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An aerogel comprising a mixture of nanofibers and microfibers.

2. The aerogel of claim 1, wherein said aerogel further comprises a binder.

3. The aerogel of claim 2, wherein said binder is selected from the group consisting of gelatin, collagen, gelatin methacryloyl, agarose, poly(organophosphazenes), sodium alginate, hyaluronic acid, fibrinogen, fibronectin, laminin, poly(N-isopropylacrylamide), poly(vinyl methyl ether), poly(ethylene glycol), poly(propylene glycol), poly(methacrylic acid), poly(vinyl alcohol), chitosan, starch, pectin, cellulose, methylcellulose, sodium polyacrylate, and starch-acrylonitrile co-polymers.

4. The aerogel of claim 2, wherein said binder is gelatin.

5. The aerogel of claim 2, wherein said nanofibers and microfibers are coated with said binder.

6. The aerogel of claim 2, wherein said nanofibers and microfibers and said binder are crosslinked.

7. The aerogel of claim 6, wherein said nanofibers and microfibers and said binder are crosslinked by glutaraldehyde.

8. The aerogel of claim 1, wherein the average diameter of said microfibers is about 10 to about 100 times greater than the average diameter of said nanofibers.

9. The aerogel of claim 1, wherein the ratio of nanofibers to microfibers is from about 25:75 to about 75:25.

10. The aerogel of claim 1, wherein said nanofibers have an average diameter from about 100 nm to about 500 nm;
wherein said microfibers have an average diameter from about 1 μm to about 30 μm;
wherein said nanofibers have an average length of less than about 250 μm; or
wherein said microfibers have an average length less than about 10 mm.

11. The aerogel of claim 1, wherein said nanofibers have an average diameter from about 100 nm to about 500 nm; wherein said microfibers have an average diameter from about 1 μm to about 30 μm; wherein said nanofibers have an average length of less than about 250 μm; and wherein said microfibers have an average length less than about 10 mm.

12. The aerogel of claim 1, wherein said nanofibers comprise a first polymer and said microfibers comprise a second polymer.

13. The aerogel of claim 12, wherein said first polymer and said second polymer are different and/or wherein said first polymer an/or second polymer are hydrophobic polymers.

14. The aerogel of claim 1, wherein said nanofibers and microfibers comprise polycaprolactone (PCL).

15. The aerogel of claim 12, wherein said nanofibers and/or said microfibers further comprise a poloxamer.

16. The aerogel of claim 1, wherein said nanofibers comprise electrospun nanofibers and/or said microfibers comprise wet spun microfibers.

17. The aerogel of claim 1, wherein said aerogel further comprises a drug, bioactive agent, polypyrrole, magnetic nanoparticle, cells and/or tissue.

18. A method of synthesizing the aerogel of claim 1, said method comprising freeze drying a composition comprising said nanofibers and microfibers.

19. A method of treating an injury or wound, treating hemorrhage, and/or regenerate tissue in a subject, said method comprising applying or administering an aerogel of claim 1 to said subject.

20. A method of collecting a biological sample from a subject, said method comprising contacting an optionally compressed aerogel of claim 1 to said subject.
